(19) United States
(12) Patent Application Publication
(10) Pub. No.: US 2020/0085705 A1
(43) Pub. Date: Mar. 19, 2020

(54) Method of Encapsulating Compounds

(71) Applicant: AGENCY FOR SCIENCE, TECHNOLOGY AND RESEARCH, Singapore (SG)

(72) Inventors: Agata Maria Brzozowska, Singapore (SG); Maria N. Antipina, Singapore (SG)

(21) Appl. No.: 16/469,758

(22) PCT Filed: Dec. 22, 2017
(86) PCT No.: PCT/SG2017/050639

§ 371 Date: Jun. 14, 2019

(30) Foreign Application Priority Data
Dec. 22, 2016 (SG) 10201610757Q

(51) Int. Cl. A61K 8/11; A61K 8/90; A61K 9/50; B01J 13/10

(52) U.S. Cl. CPC A61K 8/11; A61K 8/90; A61K 9/5031; A61K 2800/594; A61K 2800/10; A61K 2800/56; B01J 13/10

(57) ABSTRACT

The present invention relates to a method of encapsulating an uncharged, soluble, active compound within a self-assembled ionomer complex formed from polyelectrolytes, wherein the said method comprises the steps of mixing a solution comprising at least one polycation and polyanion with active compound, or adding a polyion to a solution comprising an oppositely charged polyion coupled to the active compound, wherein the interaction between the active compound and ionomer complex is non-ionic and non-covalent in nature. The invention also relates to an ionomer complex prepared thereform for use in transport and delivery of the compounds. In a preferred embodiment, the ionomer complex comprises poly-L-arginine, Poly(ethylene oxide)-b-poly(acrylic acid) (PAA-b-PEO), wherein the said ionomer encapsulates 4-methoxyphenyl β-D-glucopyranoside (MG).

### TECHNICAL FIELD

The present invention generally relates to methods of encapsulating small molecules which are water soluble, uncharged and/or non-ionic. The present invention also relates to ionomer complexes formed using the disclosed methods.

### BACKGROUND ART

Encapsulation of active compounds for enhancing their bioavailability is an issue of interest, particularly in the field of biomedicine, cosmetics and personal care industry. There are existing solutions for encapsulation of charged hydrophilic, hydrophobic and amphiphilic compounds. However, these solutions are not well suited to encapsulate small and uncharged compounds. The known methods encapsulate the compounds by covalent and/or ionic bonding and this alters the original chemical structures, functionalities and/or activities of the encapsulated compounds and this is not desirable when the compounds need to be delivered or transported with no change in these chemical properties.

Another difficulty of encapsulating active compounds relates to the molecular mass of the hydrophilic compound to be encapsulated. Generally, the smaller the molecular mass of the molecule, the greater the difficulty to encapsulate it effectively. The existing methods for encapsulation of water soluble products require formation of complex, multi-phase systems (emulsions) and this decreases loading efficacy and increases the complexity and costs of the encapsulation process. For instance, encapsulation of low molecular weight, uncharged and water soluble compounds are typically achieved using oil-in-water emulsions. A second phase—oil phase needs to be introduced to form an emulsion. While cost-effective and well-studied, this method is not ideal because of the need to introduce an oil phase into the system. This necessarily results in a further step to subsequently remove the oil in order to recover the compound of interest.

The formation of water in oil emulsion is necessary to prevent the encapsulated molecule from diffusing through the layer of oil phase surrounding the aqueous phase with the compound. The concentrated aqueous solution is emulsified, i.e. split into tiny droplets surrounded by layer of oil and subsequently a capsule is built around such droplets.

The water soluble, uncharged and small molecules can diffuse out from the capsule during the washing process due to concentration gradient that exists across-the capsule wall, i.e. high concentration inside and low concentration outside the capsule wall. Hence, the existing methods require formation of water in oil emulsion so that the molecule cannot diffuse through the layer of oil phase surrounding the aqueous phase with the compound. The concentration gradient is also a reason why, the concentration inside the capsule can never be higher than the concentration in the surrounding solution.

Hence, there is a need to provide an alternative method for encapsulation of small, uncharged and hydrophilic compounds. In particular, it is desired to provide a method of coupling or attaching such compounds with a reasonable loading efficiency and without altering the chemical and/or therapeutic properties of the compound. More particularly, it is desired to provide a method of encapsulating such compounds in a straightforward manner that obviates the need for using multi-phase emulsion systems.

### SUMMARY OF INVENTION

According to one aspect of the present invention, there is provided a method of coupling an active compound to an ionomer complex, the method comprising at least one step selected from: (a) mixing a solution comprising at least one polycation and at least one polyanion, with the active compound; or (b) adding the polycation to a solution comprising the polyanion that is coupled to the active compound; or (c) adding the polyanion to a solution comprising the polycation that is coupled to the active compound; to thereby form the ionomer complex having the active compound encapsulated therein; wherein the active compound is uncharged and water soluble and of low molecular weight; wherein the interaction between said active compound and the ionomer complex is non-ionic and non-covalent in nature.

In another aspect, there is provided an ionomer complex comprising at least one active compound coupled thereto, wherein said active compound is uncharged and water-soluble, wherein the active compound is coupled to the ionomer complex via a non-ionic and non-covalent interaction.

It has been surprisingly found that the disclosed method is particularly useful for enclosing uncharged and water-soluble compounds. It has been further surprisingly found that the disclosed polyelectrolyte ionomer complexes are able to encapsulate these compounds in a stable manner, without requiring any ionic and/or covalent interactions between the compound being encapsulated and the ionomer complex. Advantageously, the present disclosure provides, for the first time, the ability to store, transport and deliver these small, uncharged compounds without altering their original chemical structures, functionalities and/or activities.

Further advantageously, the disclosed method provides a straightforward and spontaneous way of encapsulating the compound in a single mixing step, with substantial flexibility. For instance, the method may comprise mixing each of the polyanions, polycations and the active compound simultaneously. In other embodiments, the active compound may be first coupled to one of the polyions, prior to mixing with the oppositely charged polyion. It has been found that each of these disclosed embodiments result in the formation of an ionomer complex encapsulating one or more of these uncharged compounds.

Still advantageously, the disclosed method does not require the use of multiphase oil-in-water or water-in-oil emulsions; and thus does not require separate steps of removing the oil and recovering the active compound.

### Definitions

The following words and terms used herein shall have the meaning indicated:

The term “uncharged”, when used herein to describe a compound or a molecule, means that the compound or molecule expresses no charges at all, whether positive or negative. In embodiments, this could mean that the compound or molecule comprises no charged ions or species. In another embodiment, an uncharged molecule is a molecule that has no net charge. There may be, however, uneven distribution of electrons around the atoms within the molecule—some atoms will have more electrons and some atoms will have less. Accordingly, an uncharged compound may include a polar compound.

The term “encapsulation” as used herein refers to an uncharged active compound that is in the vicinity of an ionomer complex formed by mixing polyelectrolytes of opposing charges wherein the interaction between the compound and the complex consists of at least hydrogen bonding, hydrophobic interaction, Lennard Jones interaction, Van der Waals forces or a combination of any of the above.

The terms “coupled”, “bonded”, “attached” or “encapsulated”, when used in the context of the present invention are used to describe an interaction between the active compound and the ionomer complex, and are intended to mean that the interaction is non-covalent and non-ionic in nature.

The term “ionomer complexes (ICs)” as used herein, may also be known as “complex coacervate core micelles” or “polyion complex micelles”, are spontaneously formed upon mixing at least two, water soluble, oppositely charged polyelectrolytes, of which at least one has an uncharged block attached, at stoichiometric charge ratio. A driving force for the formation of ICs is electrostatic attraction between the charged species and entropy gain upon release of counter ions. In one embodiment, the charged blocks form structure that be called “core’ (coacervate phase) and the neutral block(s) form structure that may be called “corona” which stabilize the ICs in a solution. This is a “special case” of complex coacervate core micelles where linear polyelectrolytes of specific ratio between lengths of the blocks are used. The formation of a core-corona structure in ICs is dependent on the components used during the preparation of the ICs and may not be restricted to having a core-corona structure. In another embodiment, the polyelectrolytes and the uncharged compound may form aggregates which may or may not have well-defined morphologies.

The expression “functional group capable of forming hydrogen bonds” as used herein, may refer to a functional group that can react as a hydrogen bond acceptor, a hydrogen bond donor or a combination of both. International Union of Pure and Applied Chemistry (IUPAC) defines hydrogen bond as “A form of association between an electronegative atom and a hydrogen atom attached to a second, relatively electronegative atom. It is best considered as an electrostatic interaction, heightened by the small size of hydrogen, which permits proximity of the interacting dipoles or charges.” A compound to be encapsulated may comprise more than one functional group for forming hydrogen bonds. For example, using glycine as an example compound to be encapsulated in the ionomer complex, i.e., chemical formula HOOCCH₂NH₂, the amino group and the carboxylic acid group provide two groups for hydrogen bonding.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF EMBODIMENTS

Exemplary, non-limiting embodiments of a method for coupling to an active compound will now be disclosed.

In one embodiment, there is disclosed a method of coupling an active compound to an ionomer complex (thereafter termed as IC-MG), the method comprising at least one step selected from: (a) mixing a solution comprising at least one polycation and at least one polyanion, with the active compound; or (b) adding the polycation to a solution comprising the polyanion that is coupled to the active compound; or (c) adding the polyanion to a solution comprising the polycation that is coupled to the active compound; to thereby form the ionomer complex having the active compound encapsulated therein; wherein the active compound is uncharged and water soluble; wherein the interaction between said active compound and the ionomer complex is non-ionic, and non-covalent in nature.

The interaction between the active compound and the ionomer complex may be selected from the group consisting of: hydrogen bonding, hydrophobic interactions, Lennard Jones interactions, and Van der Waals forces. In an embodiment, the active compound may be coupled to the ionomer complex via hydrogen bonding. Advantageously, hydrogen bonds can be readily formed by bonded atoms having a difference in their electronegativities, which results in the formation of a weak electrostatic force exerted by the functional group comprising said bonded atoms.

The active compound may have an atomic mass of less than 1 kDa. For instance, the active compound may have a molecular weight of less than about 1000 Da, 950 Da, 900 Da, 850 Da, 800 Da, 750 Da, 700 Da, 650 Da, 600 Da, 550 Da, 500 Da, 450 Da, 400 Da, 350 Da, 300 Da, 250 da, 200 Da, 150 Da or 100 Da, or may be in a range comprising an upper limit and a lower limit selected from any two values from the above. The encapsulation of low molecular weight compounds is a challenging endeavor. The presently disclosed method is surprisingly capable of encapsulating compounds that have an atomic mass of less than 1 kDa. In one embodiment, the disclosed method is capable of encapsulating a compound of less than 500 Da. In yet another embodiment, the disclosed method is capable of encapsulating a compound of less than 350 Da.

In the disclosed method, at least one of the polycation or the polyanion may be a block co-polymer comprising a neutral block. In the block copolymer, the ratio of neutral repeating units to charged repeating units may be about 1:1, about 2:1, about 3:1, about 4:1, about 5:1, about 1:2, about 1:3, about 1:4 or about 1:5. In one embodiment, the ratio of neutral repeating units to charged repeating units in the block polymer may be about 1:1. Advantageously, the use of a polycation or a polyanion which is a block co-polymer having a neutral block facilitates the formation of the ionomer complex.

In another embodiment, the polyanion may be PAA₁₀₄-b-PEO₅₁₀ where PAA is the anionic block and PEO is the neutral block. Hence, the ratio of the neutral blocks to the polyanion blocks is 510:104 which is about 5. The presence of the neutral blocks is responsible for the formation of the ionomer complexes that stay in solution and have a finite size, i.e., they grow up to a certain size and do not aggregate further. The neutral blocks should be of sufficient length to stabilize the complex. The formation of complexes is driven by interactions between the polyanion and the polycation blocks (complex coacervation). In the absence of the neutral blocks, there will be no aggregates but rather a separate layer of complex coacervate.

Further, in the disclosed method, the ionomer complex may comprise one or more functional groups selected from the group consisting of: an alcohol group, a carbonyl group, an ether group, an ester group, a carboxylic acid group, an amine group, an amide group, a carbamide group, an imine group, an imino group, an imidazole group, a guanidine group, a fluoro group and a cyano group, and wherein said one or more functional groups is coupled to the active compound by the non-ionic and non-covalent interaction. These functional groups may be located on a neutral region of the ionomer complex and/or the charged region of the ionomer complex. These functional groups may be positioned in a sterically unhindered location of the ionomer complex to enable the formation of said interactions. Advantageously, the availability and distribution of the functional groups may allow plural molecules of the active compound to be substantially dispersed throughout the structure of the ionomer complex. The functional groups also allow the active compound to be substantially immobilized on the ionomer complex to thereby facilitate its transport and delivery.

The polyion of choice needs to be suitable for use in a given application. It may be selected from the group comprising proteins or synthetic polyelectrolytes. The composition of the ionomer complexes may not be restricted to any kind of polyions provided the polyions can participate in nonionic interactions and are appropriate to the application of interest, e.g., the polyions need to be approved for consumption if it were to be used in food products; need to be non-toxic if it were to be used for applications in the marine environment, etc.

The ionomer complex may comprise one or more polyanions selected from the group consisting of: polyacrylic acid, polymethacrylic acid, polystyrene sulfonate, polyphosphoric acid, polyglutamic acid and polyaspartic acid.

The ionomer complex may comprise one or more polycations selected from the group consisting of: poly-L-arginine, poly-D-arginine, poly-L-tryptophan, poly-D-tryptophan, poly-L-histidine, poly-D-histidine, a—poly-L-lysine, ε-poly-L-lysine, α-poly-D-lysine and ε-poly-D-lysine.

The ionomer complex may comprise one or more neutral polymer blocks selected from the group consisting of: polyethylene oxide (PEO), polytyrosine, polylactic acid, polycaprolactone, polyurethanes or polyanhydrides.

In the disclosed method, the uncharged active compound to be encapsulated may contain at least one functional group capable of forming hydrogen bonds accordingly to the IUPAC definition describe herein, where the functional group may be independently selected from an alcohol group, a carbonyl group, an ether group, an ester group, a carboxylic acid group, an amine group, an amide group, a carbamide group, an imine group, an imino group, an imidazole group, a guanidine group, a fluoro group, a cyano group or a combination of any of the above.

The uncharged compound to be encapsulated by the ionomer complex (IC) must be water soluble and may participate in hydrophobic interaction, Lennard Jones interaction and Van der Waals forces with the IC. Advantageously, the uncharged compound is water soluble which means that it can be encapsulated and be transported in an aqueous environment. This would find useful applications in a field where a particular active compound needs to be transported from one point to another in an aqueous phase, e.g., biomedicine and cosmetics.

The polyelectrolyte used for preparing the ionomer complex must be water soluble and may comprise at least one functional group capable of forming hydrogen bonds accordingly to the IUPAC definition describe herein, where the functional group is independently selected from an alcohol group, a carbonyl group, an ether group, an ester group, a carboxylic acid group, an amine group, an amide group, a carbamide group, an imine group, an imino group, an imidazole group, a guanidine group, a fluoro group, a cyano group or a combination of any of the above. The hydrogen bond forming group may be found to reside in any part of the polyelectrolyte. In one embodiment, the hydrogen bond forming group may reside on the cation of the polyelectrolyte. In another embodiment, the hydrogen bond forming group may reside on the anion of the polyelectrolyte. In yet another embodiment, the hydrogen bond forming group may reside on the neutral portion of the polyelectrolyte. In yet further another embodiment, the hydrogen bond forming group may reside on the backbone of the polyelectrolyte.

The polyelectrolyte used for preparing the ionomer complex may comprise a cationic group (may be referred to as the polycation) wherein the cationic group is selected from an ammonium cation, an iminium cation, an amidinium group, an imidazolium cation, a guanidinium cation, a pyridinium cation, or a combination of any of the above.

The polyelectrolyte used for preparing the ionomer complex may comprise an anionic group (may be referred to as the polyanion) wherein the anionic group is selected from a carboxylate anion, a hydroxyl anion, a fluoride anion, a chloride anion, a bromide anion, an iodide anion, an oxide anion, a carbonate anion, a sulphate anion, a nitrate anion, a sulphide anion, a phosphate anion, or a combination of any of the above.

The polycation or the polyanion may be linked to a neutral polymer block which does not contain any charges, i.e., absence of cations, anions or a combination of both. The polycation, polyanion or the neutral polymer may also be selected to be biodegradable.

The polycation block, the polyanion block and the neutral polymer block selected for forming an IC may independently have a molecular weight of about 1.0 kDa, 1.5 kDa, 2.0 kDa, 2.5 kDa, 3.0 kDa, 3.5 kDa, 4.0 kDa, 4.5 kDa, 5.0 kDa, 5.5 kDa, 6.0 kDa, 6.5 kDa, 7.0 kDa, 7.5 kDa, 8.0 kDa, 8.5 kDa, 9.0 kDa, 9.5 kDa, 10.0 kDa, 12.5 Da, 15.0 kDa, 17.5 kDa, 20.0 kDa, 22.5 kDa, 25.0 kDa, 30.0 kDa, 35.0 kDa, 40.0 kDa, 45.0 kDa, 50.0 kDa, 55.0 kDa, 60.0 Da, 65.0 kDa, 70.0 kDa, 75.0 kDa, 80 kDa, 90 kDa or 100 Da, or may be in a range comprising an upper limit and a lower limit selected from any two values from the above.

In one embodiment, the polycation used for preparing the ionomer complex may comprise a guanidinium cation. In another embodiment, the polycation may be poly-L-arginine (PArg) having a molecular weight of more than 70 kDa.

In another embodiment, the polyanion used for preparing the ionomer complex may comprise a carboxylate anion. In another embodiment, the polyanion may be poly(acrylic acid)-b-poly(ethylene oxide) (PAA-b-PEO) having a molecular weight of about 7.5 kDa-b-22.5 kDa.

PArg is a biodegradable polymer approved for cell delivery. PEO (FDA, 21CFR177.1620) and PAA are polymers approved for use in biological systems. Moreover, these polymers are easily available in industrial scale which is an important consideration for practical applications of a developed method.

The ICs and the IC-MGs may be prepared by mixing at least one polycation with at least one polyanion at a pH of about 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0 or in a range wherein the upper and lower limits are selected from a combination of any of the above pH values. In one embodiment, the ICs and IC-MGs may be prepared at a pH of about 8.0. In another embodiment, the ICs are formed at a pH value where both polyelectrolytes are charged to the highest degree.

In one embodiment, the optimum pH is pH at which the complexes are stable and electrostatic interactions between ICs constituents are the strongest, i.e. majority of the chargeable groups are dissociated. This depends on the pKa of the respective polyelectrolyte. Otherwise, pH is not a critical parameter for encapsulation of uncharged compounds.

The pH of the polyelectrolyte may be pre-adjusted to 7.0 before it is used for preparing the ICs used for encapsulating the uncharged active compound. In one embodiment, the polyanion may be adjusted to pH 7.0 using 1M sodium hydroxide. In another embodiment, the polycation may be adjusted to pH 7.0 using 1M hydrochloric acid. The adjustment of the polycation and polyanion solutions to pH 7 is selected as a standard reaction condition. As mentioned, the ICs are prepared at a pH where the highest degree of dissociation of the charged groups takes place, i.e., the largest number of opposite charges available for interaction. If such a pH for the two polyelectrolytes is very far apart, a pH value that is in between these two values would be selected. Accordingly, pH=7 is usually selected.

In one embodiment, it is possible to mix two polyelectrolytes at pH=2 or 10 (extreme examples), but in such a case, one of the polyelectrolytes would be in undissociated form. In either case, there would be too few of one of the charges (either the negative charges or the positive charges) to form a complex. For example, at pH<3, PAA hardly has any charges.

In the disclosed method, the ionomer complex may be prepared in the presence of a salt solution wherein the salt may be selected from the group comprising sodium chloride, potassium chloride, ammonium chloride, sodium hydroxide, potassium hydroxide, ammonium hydroxide, sodium hydrogen carbonate, potassium hydrogen carbonate, ammonium hydrogen carbonate, sodium nitrate, potassium nitrate, ammonium nitrate, sodium sulfate, potassium sulfate, ammonium sulfate or a combination of any of the above.

For a given pair of oppositely charged polyelectrolytes, there is a range of salt concentrations beyond which the complex coacervation does not take place. However, if the salt concentration is too low, the system will flocculate or precipitate. If the salt concentration is too high, the interactions between the oppositely charged groups will be suppressed and complex coacervate phase will not take place. In some cases it would be beneficial to reach as high a physiological salt concentration. In case of high salt concentrations, it is necessary that interactions other than electrostatic interactions exist between polyelectrolytes to ensure that the complex does not fall apart. In one embodiment, it is preferable to work with monovalent salts as the monovalent ions interfere with interactions between the polyelectrolytes. Literature data and our experience show that encapsulation should be done in presence of salt.

In one embodiment, the optimum salt concentration is one at which the complex coacervation takes place and the complexes are stable and electrostatic interactions between ICs constituents are not supressed. Otherwise, the salt concentration is not a critical parameter for encapsulation of uncharged compounds.

The concentration of the salt for preparing the ICs may be in a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 11 mM, 12 mM, 13 mM, 14 mM, 15 nM, 16 mM, 17 mM, 18 mM, 19 mM, 20 nM, 21 mM, 22 mM, 23 mM, 24 mM, 25 nM, 26 mM, 27 mM, 28 mM, 29 mM, 30 nM, or may be in a range comprising an upper limit and a lower limit selected from any two values from the above. In one embodiment, the salt used is NaCl and is provided in a concentration of around 10 mM.

The preparation of the ionomer complex and/or encapsulation of an uncharged active compound within an ionomer complex according to the methods described herein may be carried out in a temperature range of about 5° C. to about 55° C., 5° C. to about 50° C., 5° C. to about 45° C., 5° C. to about 40° C., 5° C. to about 35° C., 5° C. to about 30° C., 5° C. to about 25° C., 5° C. to about 20° C., 5° C. to about 15° C., 5° C. to about 10° C., 10° C. to about 55° C., 10° C. to about 50° C., 10° C. to about 45° C., 10° C. to about 40° C., 10° C. to about 35° C., 10° C. to about 30° C., 10° C. to about 25° C., 10° C. to about 20° C., 10° C. to about 15° C., 15° C. to about 55° C., 15° C. to about 50° C., 15° C. to about 45° C., 15° C. to about 40° C., 15° C. to about 35° C., 15° C. to about 30° C., 15° C. to about 25° C., 15° C. to about 20° C., 20° C. to about 50° C., 20° C. to about 45° C., 20° C. to about 40° C., 20° C. to about 35° C., 20° C. to about 30° C., 20° C. to about 25° C., 25° C. to about 50° C., 25° C. to about 45° C., 25° C. to about 40° C., 25° C. to about 35° C., 25° C. to about 30° C., 30° C. to about 50° C., 30° C. to about 45° C., 30° C. to about 40° C., 30° C. to about 35° C. In one embodiment, the preparation of the ICs or the IC-Rs is carried out at about 20° C. to about 30° C.

The mixing time of the polyelectrolytes for preparing the ionomer complex and/or encapsulation of an uncharged active compound within an ionomer complex according to the methods described herein may vary between about 5 minutes to about 60 minutes. It may vary in a range of about 5 minutes to about 60 minutes, about 15 minutes to about 60 minutes, about 25 minutes to about 60 minutes, about 35 minutes to about 60 minutes, about 45 minutes to about 60 minutes, about 5 minutes to about 50 minutes, about 15 minutes to about 50 minutes, about 25 minutes to about 50 minutes, about 35 minutes to about 50 minutes, about 5 minutes to about 40 minutes, about 15 minutes to about 40 minutes, about 25 minutes to about 40 minutes, about 5 minutes to about 30 minutes, about 15 minutes to about 20 minutes.

In one embodiment, the length ratio between charged and neutral polyelectrolyte block may have a ratio of more than 1, or more than 2, or more than 3, or more than 4, or more than 5, or more than 6, or more than 7, or more than 8, or more than 9, or more than 10, or more than 12, or more than 15, or more than 18. Advantageously, length ratio between charged and neutral polyelectrolyte block length ratio is more than 3 for obtaining stable ionomer complexes.

In one embodiment, the exact length ratio between charged and neutral polyelectrolyte block may depend on the polyelectrolyte used for preparing the ICs. For instance, in the case where linear polyelectrolytes are used, the neutral block may be substantially longer than the charged block and there would be fewer molecules to form the single complex coacervate core micelle and this would affect the corona/core structure.

To encapsulate the uncharged active compound in the ionomer complex, the uncharged compound may be added to an ionomer complex after the complex is prepared by mixing a polycation and a polyanion. In one embodiment, an ionomer complex may be spontaneously formed by adding a polycation to another polyanion. The active compound may be encapsulated by the formed ionomer complex and the interaction between the active compound and the IC may comprise hydrogen bonding, hydrophobic interaction, Lennard Jones interaction, Van der Waals interaction or a combination of any of the above.

In another embodiment, to encapsulate the uncharged compound in the ICs, the uncharged compound may be added concurrently to the polyelectrolytes (comprising polycations or polyanions) when preparing the ionomer complex. The uncharged compound may participate in hydrogen bonding, hydrophobic interaction, Lennard Jones interaction, Van der Waals interaction or a combination of any of the above interactions with the polycation, the polyanion or both.

In another embodiment, to encapsulate the uncharged compound in the ICs, the uncharged compound may be pre-mixed with a polycation. The uncharged compound may participate in hydrogen bonding, hydrophobic interaction, Lennard Jones interaction or a combination of any of the above with the polycation. Thereafter, the mixture containing the uncharged compound and the polycation may be added to a polyelectrolyte comprising at least one polyanion. The uncharged compound may further participate in hydrogen bonding, hydrophobic interaction, Lennard Jones interaction or a combination of any of the above with the polyanion.

In another embodiment, to encapsulate the uncharged compound in the ICs, the uncharged compound may be pre-mixed with a polyanion. The uncharged compound may participate in hydrogen bonding, hydrophobic interaction, Lennard Jones interaction or a combination of any of the above interactions with the polyanion. Thereafter, the mixture containing the uncharged compound and the polyanion may be added to a polycation. The uncharged compound may further participate in hydrogen bonding, hydrophobic interaction, Lennard Jones interaction or a combination of any of the above with the polycation.

The uncharged compound may be encapsulated in any part of the ICs. The location of the uncharged compound within the ICs may be determined by the location of the functional groups in one or more polyelectrolytes forming the ICs which are able to interact with the uncharged compound. In one embodiment, if the majority of the hydrogen bonds are found in the charged block(s), the uncharged active compound may probably be bound within the ICs' charged block. In another embodiment, if the majority of the hydrogen bonds are found on the neutral block(s), the uncharged active compound may be bound within the ICs' neutral blocks. The same principle applies to other interactions (e.g., hydrophobic interactions, Lennard Jones Interactions, Van der Waals force, etc.) between the uncharged active compound and the polyelectrolyte.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1

FIGS. 1a-d illustrate several pathways for the formation of complex coacervate core micelles due to electrostatic interactions between linear polyelectrolytes. The figures illustrate a general principle behind the formation of the micelles.

FIG. 1a is a schematic diagram showing the formation of an ionomer complex from a polyanion and a polycation. The polyanion may comprise neutral polymer chains which are represented by the chains which extend out from the charged core in the ionomer complex.

FIG. 1b is a schematic diagram showing the formation of an ionomer complex with an active compound encapsulated therein, wherein the active compound is coupled to the polycation prior to reaction with the polyanion. The active compound may couple to the core of the ionomer complex when this pathway is used to encapsulate the active compound in the ICs.

FIG. 1c is a schematic diagram showing the formation of an ionomer complex with an active compound encapsulated therein, wherein the active compound, the polyanion and the polycation are mixed together simultaneously.

FIG. 1d is a schematic diagram showing the formation of an ionomer complex with an active compound encapsulated therein, wherein the active compound is coupled to the polyanion prior to reaction with the polyanion.

The active compound may couple to any part of the ionomer complex when either the pathway in (c) or (d) is used to encapsulate the active compound in the ICs.

The active compound is attached to the part of the polyelectrolyte that interacts with it most favourably. For example, if the compound can interact with the ICs only via hydrogen bonds, it will be located next to the NH-groups in poly-L-arginine chain as these groups can form strong hydrogen bonds. The exact location is system-specific and depends on the exact chemical composition of the compound and the polyelectrolytes.

FIG. 2

FIG. 2 is a number of graphs showing (a) the Dynamic Light Scattering (DLS) data which indicates the changes in light scattering intensity (I, in a.u.) normalized with total polymer concentration (c [mol/l]) in the sample, wherein I/c is the intensity of the scattered light measured at 173°, normalized with total polymer concentration in the solution; (b) zeta potential (mV); (c) hydrodynamic diameter (Dₕ, nm) of the aggregates; and (d) pH values, when a solution comprising PAA is titrated against a solution of PArg in 10 mM NaCl solutions. The horizontal dashed line indicates point of zero charge determined within experimental error. The vertical dashed line indicates the preferred micellar composition (PMC).

FIG. 3

FIG. 3 is a number of graphs showing (a) the changes in light scattering intensity (I, in a.u.) normalized with total polymer concentration in the sample, wherein I/c is the intensity of the scattered light measured at 173°, normalized with total polymer concentration (c [mol/l]) in the solution; (b) zeta potential (mV), and (c) hydrodynamic diameter (Dₕ, nm) of the aggregates, when an increasing amount of the active compound MG is being added to PAA-b-PEO, PArg and ICs respectively.

FIG. 4

FIG. 4 is a number of graphs showing (a) the changes in light scattering intensity (I, in a.u.) normalized with total polymer concentration in the sample, wherein I/c is the intensity of the scattered light measured at 173°, normalized with total polymer concentration in the solution; (b) zeta potential (mV), and (c) hydrodynamic diameter (Dₕ, nm) of the aggregates of the ICs consisting of PArg₃₃₂, and PAA₁₀₄-b-PEO₅₁₁ and ICs loaded with MG (IC-MG) at indicated weight percent in solution containing 10 mM NaCl against changes in temperature. Prior mixing, pH of solutions was adjusted to pH 7 with 1 M NaOH and/or 1 M HCl.

FIG. 5

FIG. 5 is a number of graphs showing (a) the changes in light scattering intensity (I, in a.u.) normalized with total polymer concentration in the sample, wherein I/c is the intensity of the scattered light measured at 173°, normalized with total polymer concentration in the solution; (b) zeta potential (mV), and (c) hydrodynamic diameter (Dₕ, nm) of the of ICs consisting of PArg₃₃₂, and PAA₁₀₄-b-PEO₅₁₁ and ICs loaded with MG (IC-MG) at indicated weight percent in solution containing 10 mM NaCl with respect to changes in salt concentration (NaCl). Prior mixing pH of solutions was adjusted to pH 7 with 1 M NaOH and/or 1 M HCl.

FIG. 6

FIG. 6 is a number of graphs showing (a) the changes in light scattering intensity (I, in a.u.) normalized with total polymer concentration in the sample, wherein I/c is the intensity of the scattered light measured at 173°, normalized with total polymer concentration in the solution; (b) hydrodynamic diameter (Dₕ, nm) of the aggregates; and (c) changes in pH of ICs consisting of PArg₃₃₂ and PAA₁₀₄-b-PEO₅₁₁ and ICs loaded with MG (IC-MG) at indicated weight percent in solution containing 10 mM NaCl against changes in concentration of NaOH (FIG. 6a), followed by changes in concentration of HCl (FIG. 6b).

FIG. 7

FIG. 7 is a couple of graphs showing the light scattering data from ICs and ICs-MG particles. Graph (a) shows the decay rate (Γ, Hz) and graph (b) shows the hydrodynamic radius (Rh, nm) with changes in angle of detection. The samples were prepared in 10 mM NaCl, pH 7, at PMC predetermined in FIG. 2. Content of MG was 19.2 [% w/w]. The characterization of ICs sample was done at 488 nm and characterization of ICs-MG sample at 633 nm.

FIG. 8

FIG. 8 shows ¹H NMR spectra of the ICs in comparison with PAA₁₀₄-b-PEO₅₁₁ and PArg₃₃₂; the highlighted regions show the change in chemical shifts that occur upon mixing the two polyelectrolytes. In particular, the NH-proton peak was not observed in the spectrum of ICs, suggesting that the environment surrounding the NH-proton in ICs may have changed after forming the ICs.

FIG. 9

FIG. 9 shows ¹H NMR spectra of the mixture of ICs+MG in comparison with the individual components of ICs and MG. The highlighted region corresponds to the proton peaks on the phenyl group on MG.

FIG. 10

FIG. 10 shows a NOESY NMR spectrum of the ICs+MG mixture; the circle highlights the interactions within the ICs while the square highlights the interactions involving MG. These may be intra- or extra-molecular interactions.

FIG. 11

FIG. 11 shows a COSY NMR spectrum of the MG.

FIG. 12

FIG. 12 shows a COSY NMR spectrum of ICs-MG.

FIG. 13

FIG. 13 shows an IC system with (a) and without MG molecules (b) using molecular dynamics simulation. Water molecules are omitted for clarity of the presentation.

FIG. 14

FIG. 14 is a number of graphs showing intramolecular Coulombic interaction energies within PAA-b-PEO (graphs a and b) and PArg (graphs c and d) for systems with (left) and without (right) MG obtained using molecular dynamic simulations.

FIG. 15

FIG. 15 is a number of graphs showing intramolecular Lennard-Jones interaction energies within PAA-b-PEO (graphs a and b) and PArg (graphs c and d) for systems with (left) and without (right) MG using molecular dynamic simulations.

FIG. 16

FIG. 16 is a couple of graphs showing Coulombic interactions between (a) PAA-b-PEO and (b) PArg with molecule MG using molecular dynamic simulations.

FIG. 17

FIG. 17 is a couple of graphs showing Lennard-Jones interactions between (a) PAA-b-PEO and (b) PArg with molecule MG using molecular dynamic simulations.

FIG. 18

FIG. 18 is a couple of graphs showing the number of MG molecules surrounding (a) PAA-b-PEO and (b) PArg using molecular dynamic simulations.

FIG. 19

FIG. 19 is a couple of graphs showing the number of hydrogen bonds between (a) PAA-b-PEO, (b) PArg and MG using molecular dynamic simulations.

FIG. 20

FIG. 20 is a number of graphs showing Coulombic interactions between PAA-b-PEO (graphs a and b), PArg (graphs c and d) and water using molecular dynamic simulations.

FIG. 21

FIG. 21 is a number of graphs showing Lennard-Jones interactions between PAA-b-PEO (graphs a and b), PArg (graphs c and d) and water using molecular dynamic simulations.

FIG. 22

FIG. 22 is a number of graphs showing the number of hydrogen bonds between PAA-b-PEO (graphs a and b), PArg (graphs c and d) and water for systems with (left) and without MG using molecular dynamic simulations.

FIG. 23

FIG. 23 is a number of graphs showing the number of hydrogen bonds between PAA-b-PEO and PAA-b-PEOR for systems with (a) and without (b) MG present using molecular dynamic simulations.

FIG. 24

FIG. 24 is a number of raw UV adsorption data from samples after first (graph a) and second dialysis (graphs b, c and d) during MG loading and release experiments.

In the figure:
Samples labelled with letter “a” refer to samples containing ICs and MG only.
Samples labelled with letter “b” refer to samples containing ICs, MG and Pronase.
Samples labelled with letter “c” refer to experiment blanks that do not contain ICs, but a volume of 10 mM NaCl corresponding to volume of ICs solution added to samples “a” and “b”, MG and Pronase.
Numbers 1-6 in sample labels refer to different concentrations of MG in the respective samples: 2=5.0%, 3=10.1%, 4=25.2%, 5=50.4%, and 6=100.9% of MG.
The word “Final” refers to samples after 2nd dialysis.

FIG. 25

FIG. 25 is a graph showing the release of MG from the ICs-MG loaded with 1%, 10% and 50% weight of MG, respectively. “Bound” on the left axis refers to the amount of MG remaining in ICs after second dialysis. “Loaded” refers to the amount of MG added to each sample at the beginning of each experiment, expressed as weight percentage of total polymer in solution.

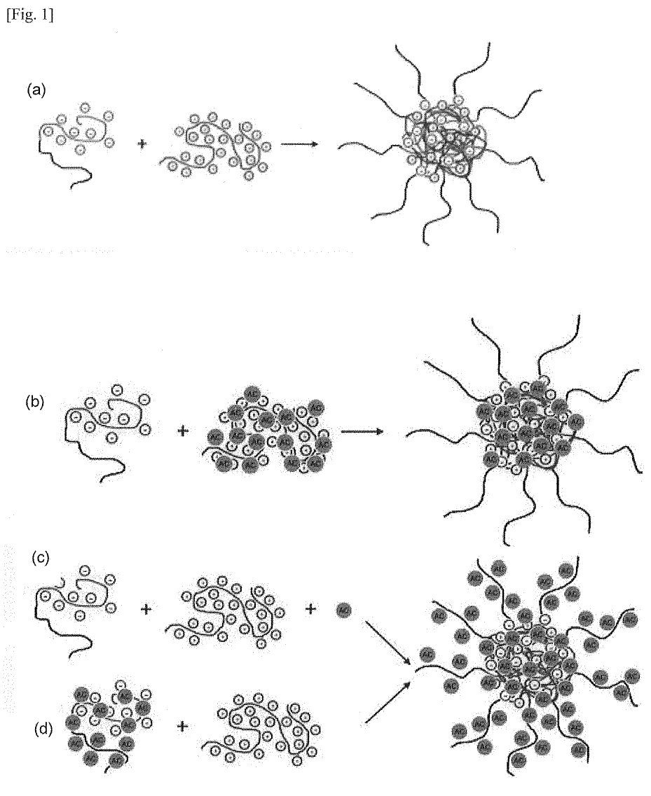

### EXAMPLES

Non-limiting examples of the invention and a comparative example will be further described in greater detail by reference to specific Examples, which should not be construed as in any way limiting the scope of the invention.

### Materials and Methods

Poly(ethylene oxide)-b-poly(acrylic acid) (PAA₁₀₄-b-PEO₅₁₁, P11302B-EOAA, Mw=22.5-b-7.5 kDa) was purchased from Polymersorce Inc. Poly-L-arginine hydrochloride (PArg₃₃₂, P3892-100MG, Mw=70-150 kDa), 4-methoxyphenyl β-D-glucopyranoside (MG, 772313-5G, Mw=286.28 Da), deuterium oxide (661643 Aldrich, Deuterium oxide, 99.99 atom % D (for D₂O), containing 1% DSS-d₆), sodium hydroxide (NaOH), hydrochloric acid (HCl), and sodium chloride (NaCl) were purchased from Sigma-Aldrich. All chemicals were used as received. De-ionized water was used in all experiments.

NMR spectra were recorded on a JEOL ECA-II (500 MHz) spectrometer in H₂O with 2.5-5 wt % D₂O at a sample temperature of 26° C. ¹H NMR spectra were measured with water suppression (Watergate WG5 pulse sequence, δ=4.64 ppm) and were referenced to DSS (δ=0.0 ppm). Data are reported as follows: chemical shifts (δ, reported in ppm), multiplicity (s=singlet, d=doublet, t=triplet, q=quartet, br=broad, m=multiplet), integration, and coupling constants (J, reported to the nearest 0.1 Hz), assignment. COSY spectra were measured with water suppression (presaturation, δ=4.64 ppm). In both the f2 and f1—dimension the FID was apodized with a sine bell wave function. For each sample, the 90° pulse width, mixing time and relaxation delay were determined and used to acquire the 2D NOESY NMR spectrum with water suppression (Watergate). In the f2-dimension the FID was apodized with a squared cosine wave function and in the f1-dimension the cosine bell wave function was used.

Dynamic (DLS) and static (SLS) light scattering measurements were performed with Brookhaven Instruments, 488 nm and 633 nm, 15 W Ion Laser System, equipped with Avalanche Photodiode Detector; DLS measurements were conducted at 90° detection angle. The light scattering titrations were performed with Malvern Zetasizer equipped with auto-titrator, at detection angle of 173°. Samples from loading experiments were analyzed with Shimadzu UV spectrophotometer (UV-3600).

Each measurement was done at least in duplicate to ensure reproducibility of the data.

All simulations were performed on the National Supercomputing Centre in Singapore, whereas the analyses were performed on local multicore workstations. The freely available code GROMACS (Groningen Machine for Molecular Simulations) was used for both the simulations and analyses.

### Example 1—Characterization Data of PAA₁₀₄-b-PEO₅₁₁, PArg₃₃₂ and 4-methoxyphenyl β-D-glucopyranoside (MG)

For PAA₁₀₄-b-PEO₅₁₁, the number 104 and 511 refer to the number of monomers in each chain. It is calculated using the given molecular weight of the whole chain divided by the molecular weight of a monomer rounded down to the nearest integer. For example, molecular weight of PAA=7.5 kDa=7500 Da, molecular weight of AA (acrylic acid)=72.6 Da and hence, there are 7500/72.6=104 AA monomers in PAA chain. This is annotated as PAA₁₀₄. The rest of the numbers can be arrived at using the same way.

### MG:

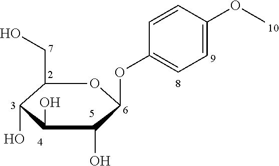

¹H NMR (500 MHz, H₂O+2.5% D₂O) δ 7.12 (d, J=8.9 Hz, 2H, H8), 6.98 (d, J=8.9 Hz, 2H, H9), 4.85 (1H, H6, not observed in 1D NMR as it is in water suppression region), 3.93 (d, J=12.4 Hz, 1H, H7), 3.81 (s, 3H, H10), 3.76 (dd, J=12.4, 5.7 Hz, 1H, H7), 3.58 (dt, J=17.5, 8.6 Hz, 4H, H2, H5 and OH), 3.52 (d, J=7.8 Hz, 1H, H3), 3.48 (d, J=9.5 Hz, 1H, H4).

### PArg₃₃₂

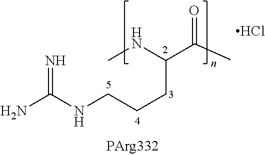

¹H NMR (500 MHz, H₂O+2.5% D₂O) δ 8.37 (br s, 0.6H, NH), 7.08 (br s, 0.3H, NH), 6.57 (br s, 0.4H, NH), 4.26 (br s, 0.2H, H2), 3.11 (t, J=6.2 Hz, 2H, H5), 1.69 (m, 2H, H3), 1.54 (m, 2H, H4).

PAA₁₀₄-b-PEO₅₁₁

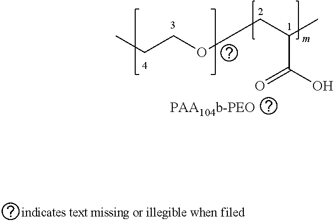

¹H NMR (500 MHz, H₂O+5% D₂O) δ 3.92-3.46 (m, 21.5H, H3 and H4), 3.38-3.30 (m, 0.46H, H1′), 2.23-1.90 (m, 1.40H, H1), 1.81-1.23 (m, 2.23H, H2), 1.17 (t, J=7.6 Hz, 0H), 1.04 (d, J=11.7 Hz, 0.66H).

### Example 2—Formation of Ionomer Complexes Loaded with an Active Compound

The following example describes the preparation of an ionomer complex (IC) according to the present invention, and its use in the encapsulation of a small, active compound 4-methoxyphenyl β-D-glucopyranoside (MG).

The ICs were formed upon mixing poly-L-arginine (Mw>70 kDa, polycation) with PAA₁₀₄-b-PEO₅₁₀ (block polyanion) in an aqueous solution containing 10 mM NaCl, at stoichiometric charge ratio. The loaded ICs (ICs-MG) were formed upon mixing the ICs with MG in a solution of 10 mM NaCl at different concentrations.

The formation of ICs and ICs loaded with MG (ICs-MG) was confirmed with dynamic (DLS) and static (SLS) light scattering and NMR. The Preferred Micellar Composition (PMC) can be deduced from the observed changes of the measured parameters, i.e., maximum in scattering intensity normalized for polymer concentration (I/c), rapid change in pH upon mixing (charge regulation) as well as complete charge neutralization (Point of Zero Charge, PZC).

The composition of the system is described by f−, defined as a fraction of negatively chargeable groups [−]/([−]+[+]) in the solution. Assuming that MG is uncharged under the experimental conditions, the PMC is expected to be at f−=0.5.

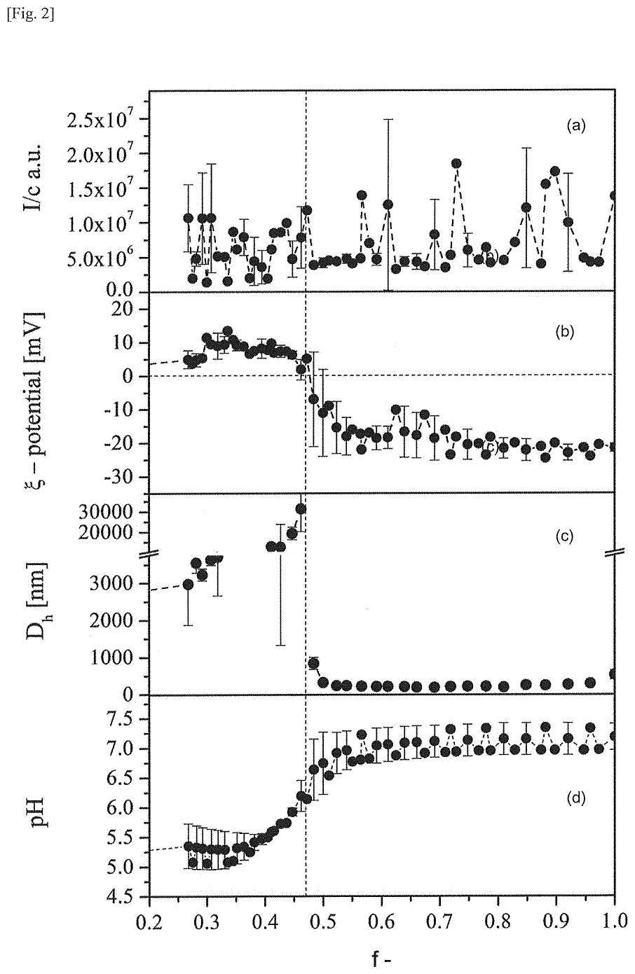

The data in FIG. 2 indicate that PMC of ICs is found at f−˜0.43 to 0.49. This apparent deviation from the expected system composition may result from the unknown exact number of chargeable groups in PArg—the number of chargeable groups is calculated based on molecular weight (Mw) being reported as “>70 kDa” for PArg, as well as steric restrictions due to branching of PArg molecules.

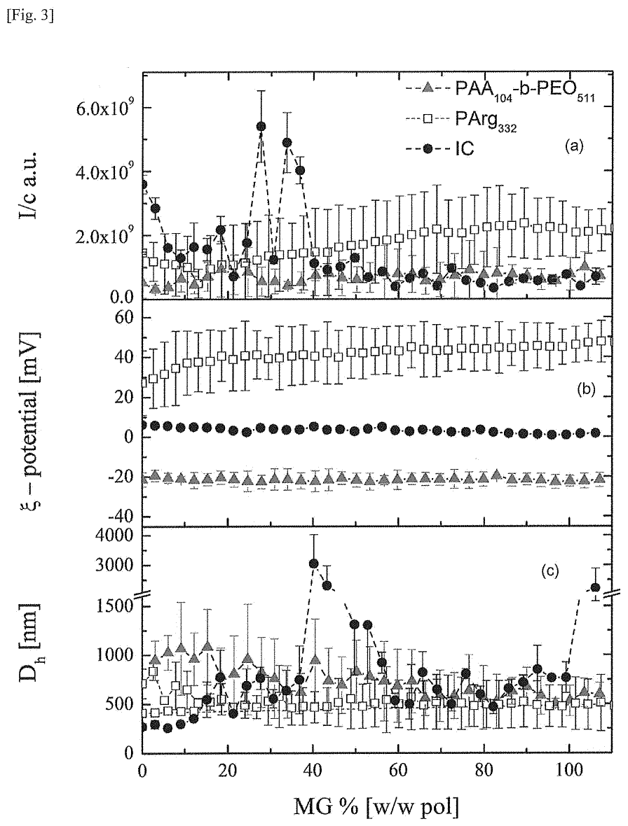

Interestingly, within experimental error, no significant change in scattering intensity was observed upon mixing the polyelectrolytes. In this case, the most accurate indication of PMC is given by system composition at PZC. It is worth mentioning, that the present data was measured within 2 minutes from addition of titrant (PArg) to the solution. Thus, taking into account the non-linearity of the respective molecules, it is possible that after some time the PMC shifts towards f−=0.5 due to repositioning of the polymer chains within the aggregates. This can be partially observed in FIG. 3. The solution of ICs was prepared at the composition derived from FIG. 2, and put aside. Titration with solution of MG was done within one day from the time of mixing the polyelectrolytes at PMC. During the experiment, we observed that the zeta potential was not zero, contrary to the results shown in FIG. 2.

### Example 3—Investigation on Changes in Hydrodynamic Diameter, Zeta Potential and Light Scattering Intensity of PArg₃₃₂, PAA₁₀₄-b-PEO₅₁₀ and ICs with Varying Concentrations of the Active Compound MG

MG was loaded into the ICs by simple mixing the respective solutions at predetermined ratios. FIG. 3 presents the results of titrations of the polyelectrolytes and ICs consisting of these polyelectrolytes with MG. Upon mixing, we observed no change in zeta potential except for slight increase in case of PArg at the beginning of titration. These results confirm that MG is not chargeable under the experimental conditions and thus cannot be bound within ICs due to ionic interactions. We observed small changes in hydrodynamic diameter (DO and normalized scattering intensity (I/c) of the aggregates, indicating bonding. This was later confirmed with NR studies.

### Example 4—Investigation on Thermal Stability of ICs and ICs-MG

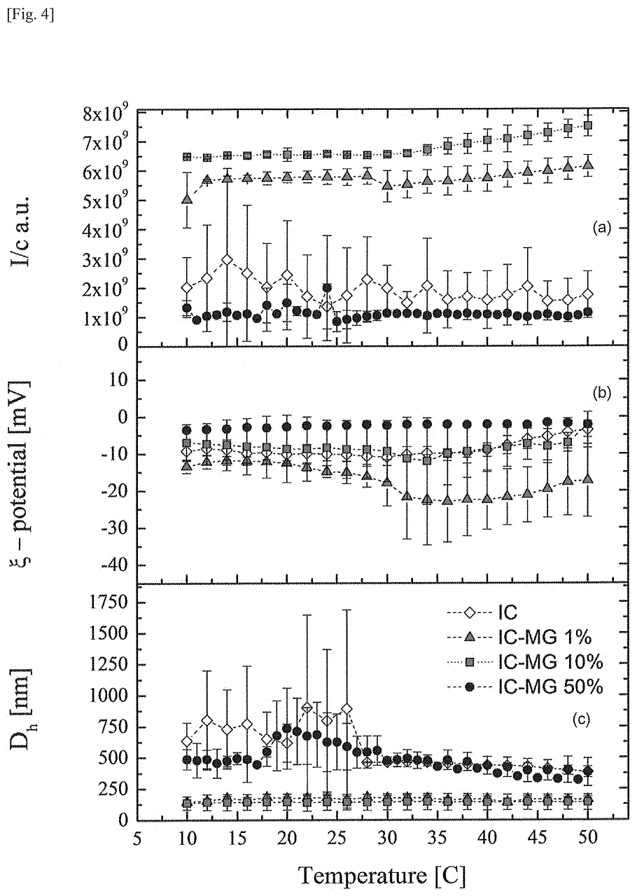

Based on the structures of the polyelectrolytes and MG, it is expected that MG would interact with the ICs via non-covalent and non-ionic interactions. The evaluation of the thermal stability of the complexes indicates that the stability of the ICs-MG increases as the content of MG increases. The experimental results can be found in FIG. 4.

The integrity of the complexes containing 50% w/w of MG as compared to others is confirmed by the stability of zeta potential, i.e., no apparent changes in the zeta potential across the temperature range tested. For ICs and ICs-MG (1% w/w and 10% w/w) decrease of the zeta potential at temperatures above 25° C. was observed. The biggest change was observed for ICs-MG (1%). Heating up resulted in oscillations in sizes of IC and ICs-MG (50%) at temperatures between 17° C. and 30° C.

Within experimental error, the scattering intensity of all samples remained unchanged during heating from 10° C. to 50° C. The results from FIG. 4 indicate stabilizing ICs structure by MG molecules.

### Example 5—Stability Studies of ICs and ICs-MG with Respect to Changes in Concentration of a Salt, a Base and an Acid

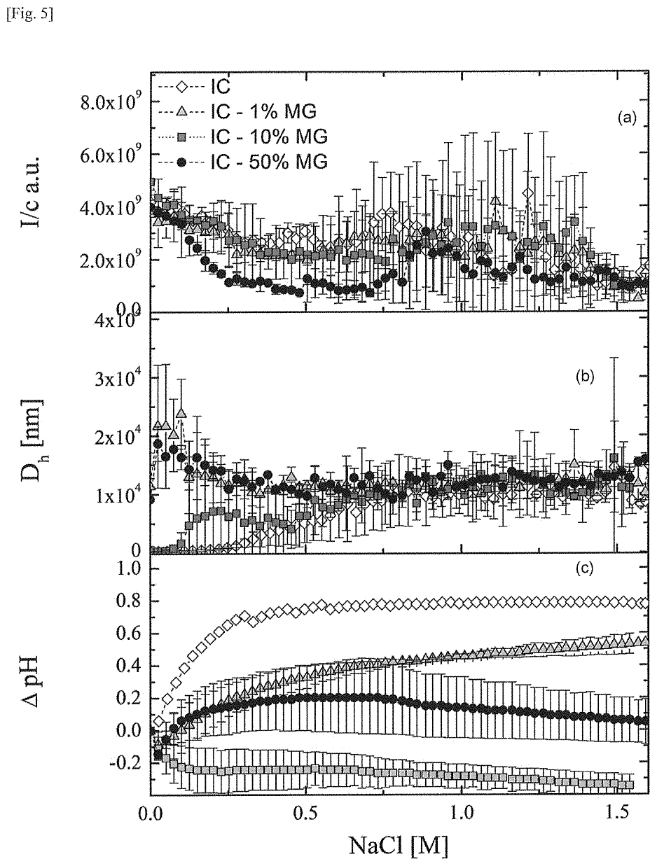

FIG. 5 shows the results of titrating ICs and ICs-MG with 5M NaCl solution. Formation of ICs results from electrostatic interactions between the oppositely charged blocks. Hence, one may expect that at sufficiently high salt concentration, the ICs may fall apart due to suppression of the electrical double layers. The effect is expected to be more pronounced in systems consisting of non-linear polyelectrolytes as compared to systems consisting of linear polyelectrolytes. The reason being that in systems consisting of non-linear polyelectrolytes, the separation between the opposite charges is greater than in case of linear polyelectrolytes due to steric restrictions.

As shown in FIG. 5, following the addition of aqueous NaCl to the respective solutions, the size of the aggregates, with exception of IC-MG 50%, increases, and scattering intensity remains relatively stable, indicating swelling of the aggregates.

Interestingly, the pH values of the respective systems also changed, i.e., pH of ICs and IC-MG 1% solutions increased, pH of IC-MG 10% solution decreased, and pH of IC-MG 50% solution initially increased followed by slow pH decrease. The change is the greatest in solution of ICs and the least in solution of IC-MG 50%. Increase in salt concentration resulted also in change of pH. The change of pH was less in solutions with highest content of MG and was the greatest in absence of MG.

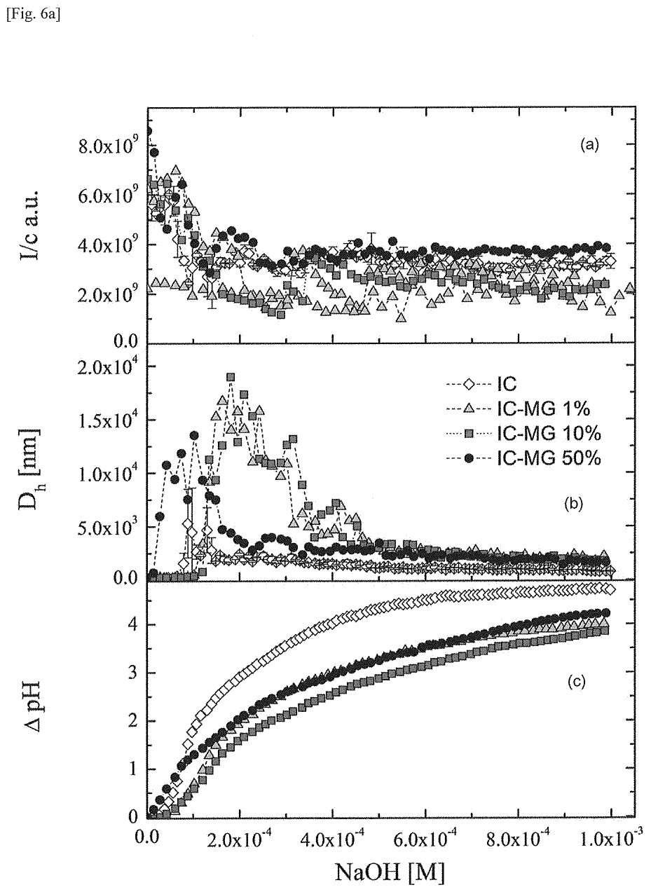

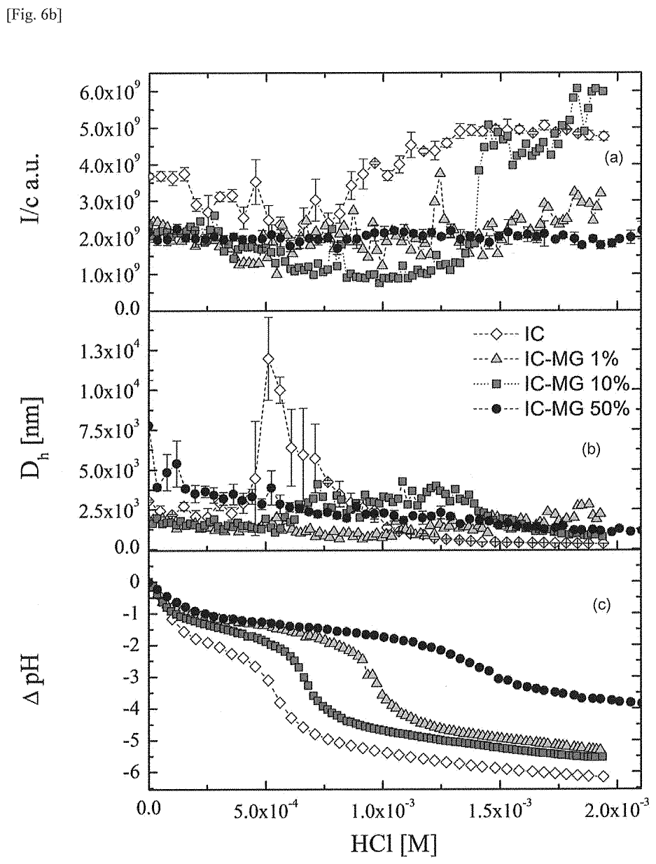

In another set of experiments, solutions containing ICs and ICs-MG were titrated with 1M NaOH followed by subsequent (the same samples) titrations with 1M HCl. The results are shown in FIGS. 6a and 6b.

Polymer concentration in each sample remained the same. Concentration of MG was 0%, 1%, 10% and 50% w/w, respectively. As concentration of NaOH in each respective solution increased, the hydrodynamic diameter (Dₕ) of all samples except ICs increased significantly and even at higher concentration of NaOH—decreased and stabilized.

The change in Dₕ was accompanied by rapid fluctuations of measured scattering intensity, except sample with 50% w/w MG indicating swelling and temporary, reversible secondary aggregation of the aggregates. The observed change of pH was the most rapid in solution without MG. In the presence of MG, pH change upon increasing the concentration of NaOH was less pronounced.

Subsequent titration with 1M HCl revealed different trend—change of pH was initially the least in ICs solution and turned rapid in solutions containing MG with the addition of HCl. As the concentration of HCl increased, pH of solutions containing MG stabilized, reaching a plateau. Further change in pH was not observed even at very high concentrations of HCl, indicating strong buffering capacity of the system. pH of ICs solution without MG was increasing with increasing concentration of HCl without producing such a pronounced plateau.

The rate of pH change varied with the concentration of the added acid, indicating strong buffering properties. The exact mechanism of the pH regulation is not yet clear.

Upon titration with HCl, following titration with NaOH to pH above pH 10, systems containing MG recovered to their initial size. The recovery (reversibility) of system without MG was less obvious. The final pH after titration with NaOH and HCl was above pH 10 and below pH 4, respectively.

### Example 6—Studies on the Structures of ICs and ICs-MG

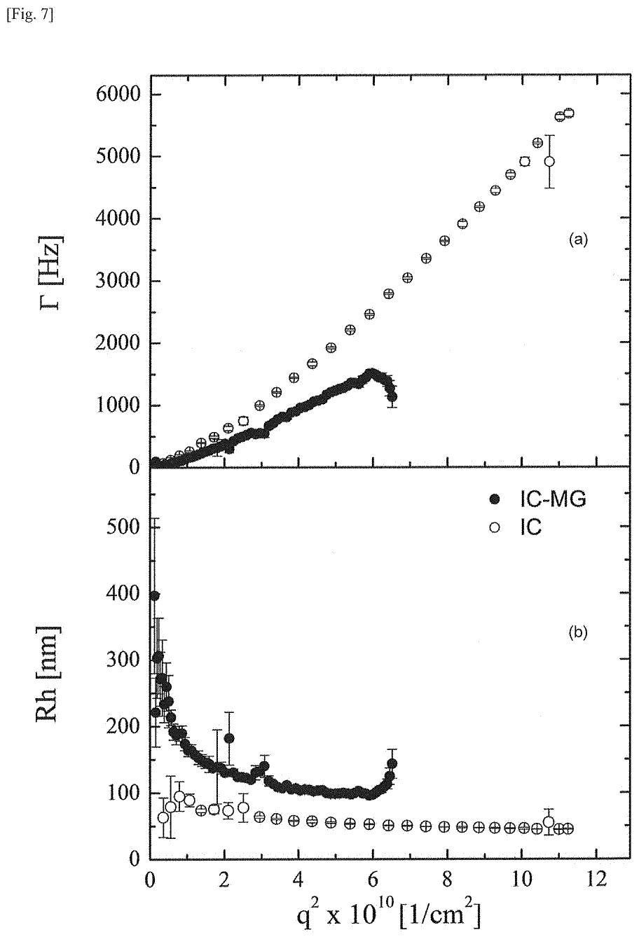

Structures of ICs and IC-MGs were studied by means of Static Light Scattering (SLS) and Nuclear Magnetic Resonance (NMR). Results of SLS experiments are shown in FIG. 7.

### Static Light Scattering Studies

SLS studies were done with Brookhaven Instruments equipped with 488 nm, 15 W Ion Laser System, Avalanche Photodiode Detector. Approximately 1 ml of sample was placed in the test vials and placed in the instrument. Intensity of scattered light was measured at various angles of detection.

FIG. 7(a) shows changes in apparent Γ (Hz), being proportional to diffusion coefficient, and FIG. 7(b) shows the changes in the hydrodynamic radius (Rh) of the aggregates with changing angle of detection (q², 1/cm²). Contrary to the Rh of ICs, the Rh of ICs-MG changes significantly with the angle of detection indicating that the morphology of the aggregates changes upon loading with MG from spherical to non-spherical.

### ¹H Proton NMR Studies of PAA-b-PEO, PArg, ICs and ICs-MG

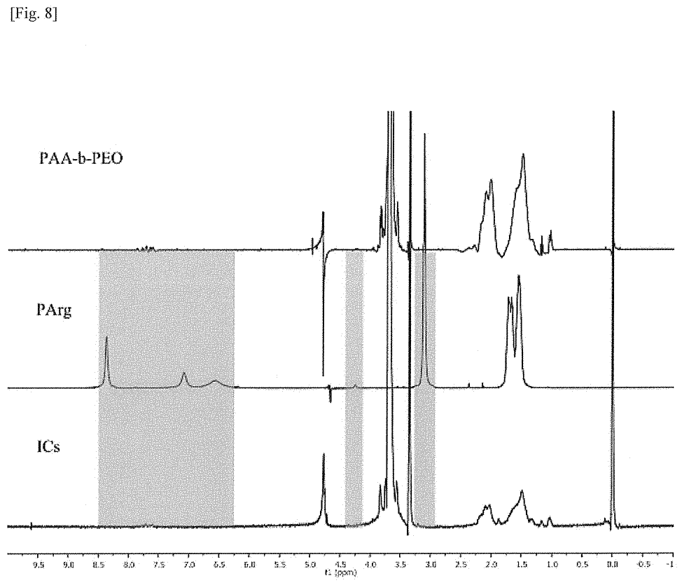

FIG. 8 shows a comparison of ¹H NMR spectra of the ICs with the individual polyelectrolytes. The key change observed is the disappearance of the PArg peaks with chemical shift δ>6 ppm; these peaks arise from the labile guanidinium NH protons, and their disappearance likely indicates a change in the hydrogen bonding environment. The change in chemical shifts of the PArg [H5] peak from 3.10 to 3.34 ppm, and the PArg [H2] peak from 4.26 to 3.73 ppm, which are on the carbon atoms adjacent to the guanidine group, also support this observation. There is no significant change in the chemical shifts of PAA₁₀₄-PEO₅₁₁ before and after the addition of PArg₃₃₂.

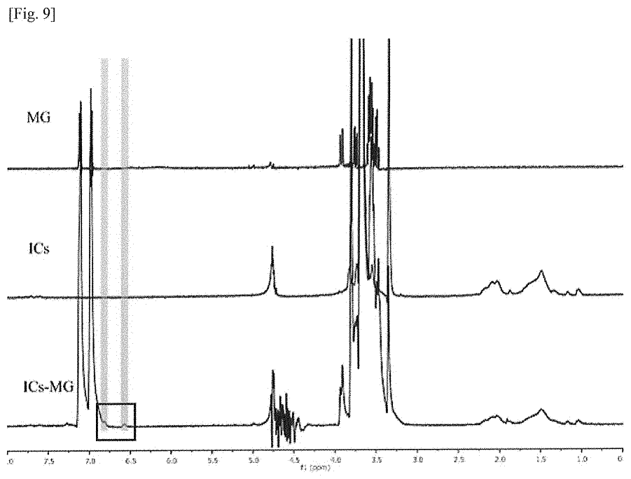

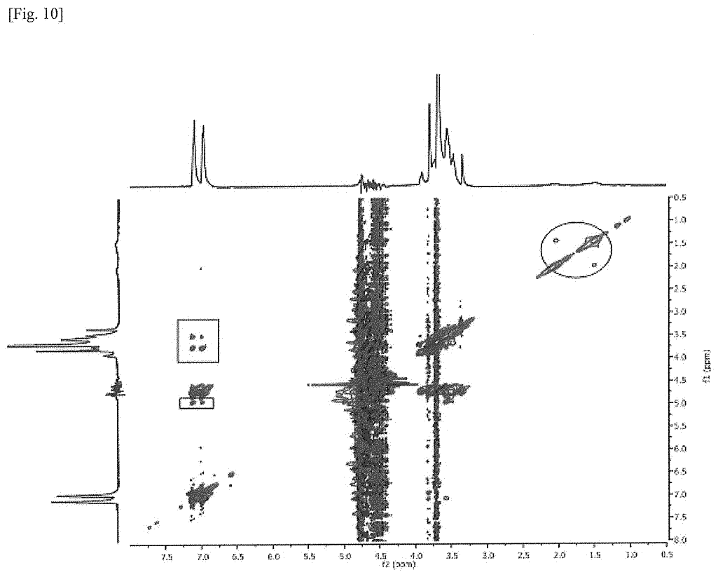

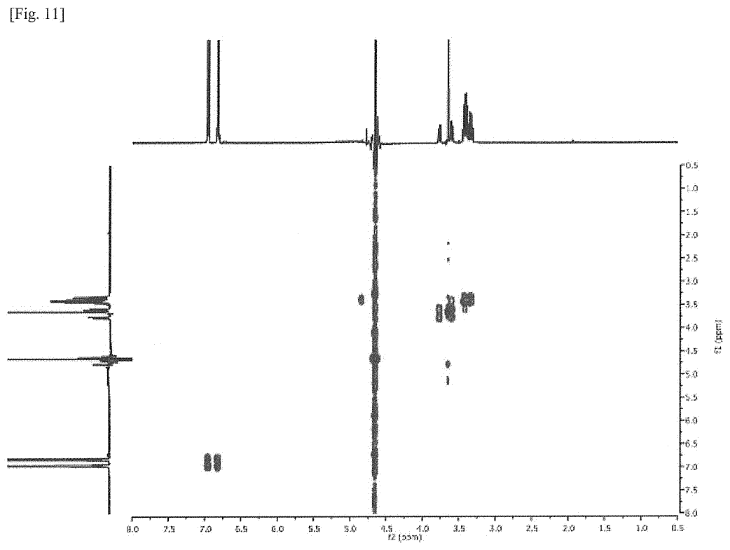

While there appears to be no significant changes in chemical shifts when MG was added to the ICs (FIG. 9), two new phenyl signals (marked within the square in FIG. 9) were observed in the ¹H spectrum of ICs+MG. These peaks were further evidenced in the COSY spectrum of this mixture (FIG. 11). Although these signals contribute <1% of the MG population, their absence in the pure MG spectrum indicate that a small percentage of MG is in a significantly different environment influenced by the ICs. Due to the small amount and peak overlap in the other spectral regions, it was not possible to detect other signals arising from this MG species.

### Nuclear Overhauser Effect Spectroscopy (NOESY—2D NMR)

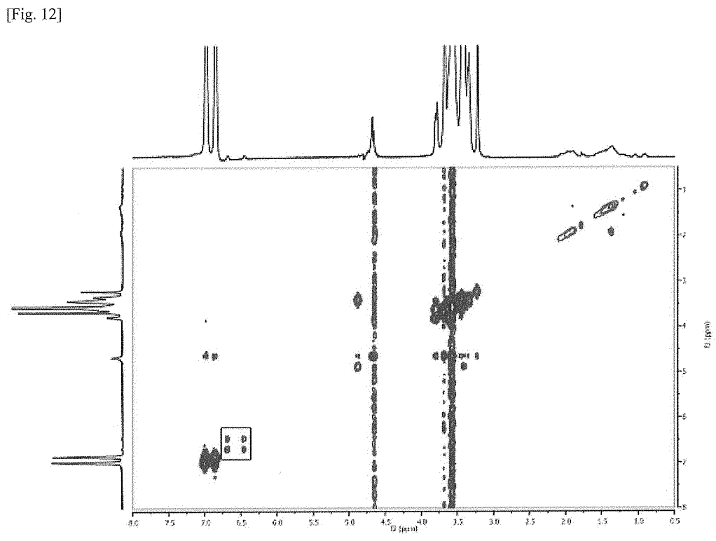

The interactions between ICs and MG were studied in more detail using Nuclear Overhauser Effect Spectroscopy (NOESY—2D NMR). This technique probes intermolecular distances by means of Nuclear Overhauser Effect (NOE), i.e., changes in resonance intensity of given proton due to saturation of the nearby proton. The results are presented as contour plot (FIG. 10), where 1D NMR spectra are plotted on vertical and horizontal axis.

NOE between two protons appear as a so-called cross-peak on the intersection of two straight lines at the chemical shifts of these protons, normal to the axis. Cross-peaks between the two protons of different chemical shifts appear off-diagonal but are symmetrical with respect to the diagonal. NOE can be observed for protons at a distance which is smaller than 5 Å from each other. A careful examination of the spectra of solutions with varying composition allowed for identification of most probable locations for interactions between components within ICs-MG.

The major difficulty with these experiments lie in that the main interaction between the ICs and MG is hydrogen bonding. These interactions cannot be probed in D₂O as deuterium replaces hydrogen in the molecules rendering the potentially interacting fragments invisible. The system under investigation consists of water soluble components that precipitate upon contact with non-aqueous solvents used in NMR measurements, e.g. chloroform.

In view of the above, the described measurements were done in water with addition of minimum D₂O required for locking (2.5-5% v/v). The water suppression was used throughout the measurements. However, the residual peaks and noise from water signal could not have been eliminated completely. Moreover, peaks associated with the components of the investigated system that have very similar shifts to water (4.64 ppm) have also been suppressed. As a result, some of the off-diagonal peaks observed in FIG. 12 do not have the expected corresponding symmetrical peaks visible. This however, should not affect the overall conclusions derived from these experiments. Based on the experimental data shown in FIGS. 8-12, inter- and intra-Nuclear Overhauser Effects were identified.

Theoretically, when the NOE is positive and gives negative NOESY cross-peaks for molecules of low molecular weight these cross-peaks are observed in the NOESY spectrum of pure. In contrast, the NOE is negative and gives positive NOESY cross-peaks for polymers and other molecules of high molecular weight; these cross-peaks are observed in the NOESY spectrum of the ICs.

Interestingly, when MG was added to the ICs, the mixture showed only positive NOESY cross-peaks. The cross-peak between δ=2.05 and 1.51 ppm is also observed in the NOESY of the ICs, as well as that of PAA₁₀₄-b-PEO₅₁₁ itself. It may arise from inter- or intramolecular NOE interactions between the polymers; however. However, it is not possible to precisely identify the interacting peaks due to peak overlap. More interestingly, positive cross-peaks from the phenyl protons of MG (δ=7.10 and 7.00 ppm) to δ=5.96 (MG), 3.76 and 3.52 ppm were observed. These cross-peaks were neither present in the COSY nor the NOESY spectra of pure MG, and cannot be explained by chemical exchange. This indicates that the strong interaction between the ICs and MG had an effect on the NOE signals of MG. Due, due to spectral overlap it is not possible to precisely identify all the cross-peaks.

### Numerical Calculations

As data obtained experimentally has not provided definitive answers about the exact nature of the observed interactions between ICs and MG, a series of numerical calculations was also performed which is aimed at resolving this issue.

In this study, molecular dynamics simulations were used to study the interactions between the polyanion (PAA-b-PEO), the polycation (PArg), the MG and the water molecules. For the purpose of this study CHARMM (Chemistry at Harvard Molecular Mechanics) and Charm Generalized Force Field parameters were used. The polypeptide PArg used in simulations was composed of 30 arginine units and its parameters were readily available in the CHARMM force field. The model block co-polymer molecule was composed of 4 acrylic acid (AA) units and 40 ethylene oxide (EO) units. The force field is needed only for the AA and EO units, as these are sufficient to assemble a co-polymer of any PAA:PEO ratio. The initial atom typing was performed using the ParaChem portal, which yielded a plausible set of parameters. A similar approach was used to parametrize the MG molecule as well.

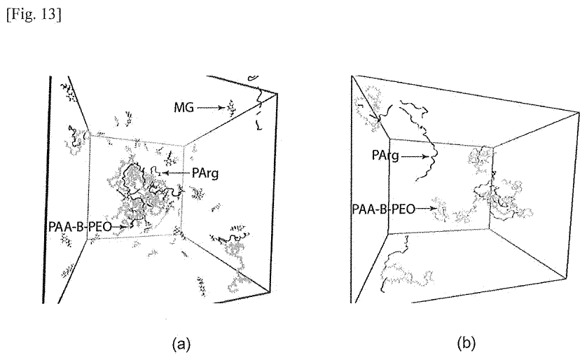

The systems were assembled by first generating an empty box that was randomly filled first with PAA-b-PEO molecules and subsequently with PArg molecules. The remaining box volume was then filled with water molecules. pH is assumed to be neutral. Overall, two kinds of systems were assembled: one with MG molecules and another one without. The system with MG molecules was generated by randomly replacing molecules of water with MG molecules. The system size was 16 nm×16 nm×16 nm and contained approximately 410,000 atoms. Chlorine ions were added to achieve electro neutrality, which is in agreement with the experimental setup, where NaCl was used as a background electrolyte. The exact system composition is summarized in Table 1, and both systems are visualized in FIG. 13.

TABLE 1
Composition of systems used in simulations

| Component | System with MG | System without MG |
|---|---|---|
| PArg | 10 | 10 |
| PAA-b-PEO | 10 | 10 |
| MG | 50 | n.a. |
| Water | 130126 | 130843 |
| Cl− | 200 | 200 |

After the assembly, the system was equilibrated by cycle of energy minimization, molecular dynamics at constant temperature (NVT ensemble) and molecular dynamics at constant temperature and pressure (NPT ensemble) with the movement of the solutes restrained. The restraints were later removed and the equilibration cycle was repeated. This approach ensured the systems are well equilibrated and any bad contacts between the solutes themselves and solute-solvent were removed. For the production run, we performed four independent runs for each system to ensure the phenomena observed are relevant and reproducible. We ran the production runs for 100,000,000 steps with a time step set to 0.002 fs, which means the total simulation time was 200 ns. The temperature was set to 300 K and pressure was set to 1 bar to mimic the experimental conditions. The long-range electrostatic interactions were evaluated using the Periodic Mesh Ewald. Periodic boundary conditions were used as well.

After the simulations, several analyses were performed to quantify the observed behavior and to determine differences between the systems with and without the MG molecule. The analyses include: interaction energies between the various solutes and solutes-solvent, radial distribution function plots for the solutes, contact and hydrogen bonding analysis, surface properties of the solutes were evaluated as well, number of water molecules around the solutes, solute properties. Unless otherwise stated the analyses were performed for the whole trajectory.

Interaction energies were computed for the following pairs of molecules PAA-b-PEO-PAA-b-PEO, PArg-PArg, PAA-b-PEO-PArg, PAA-b-PEO-MG, PArg-MG, PAA-b-PEO-Water, PArg-Water and MG-Water. Both the Coulombic and Lennard-Jones types of interactions were computed.

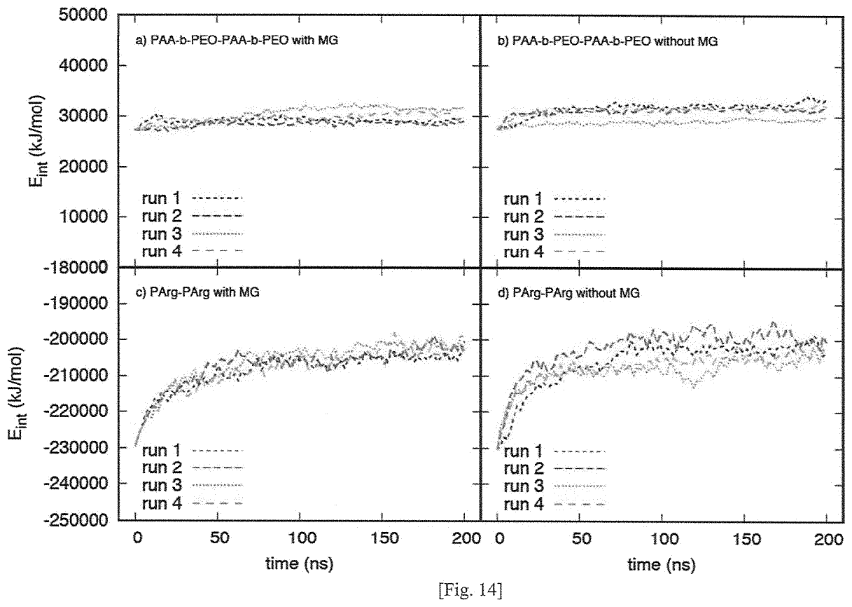

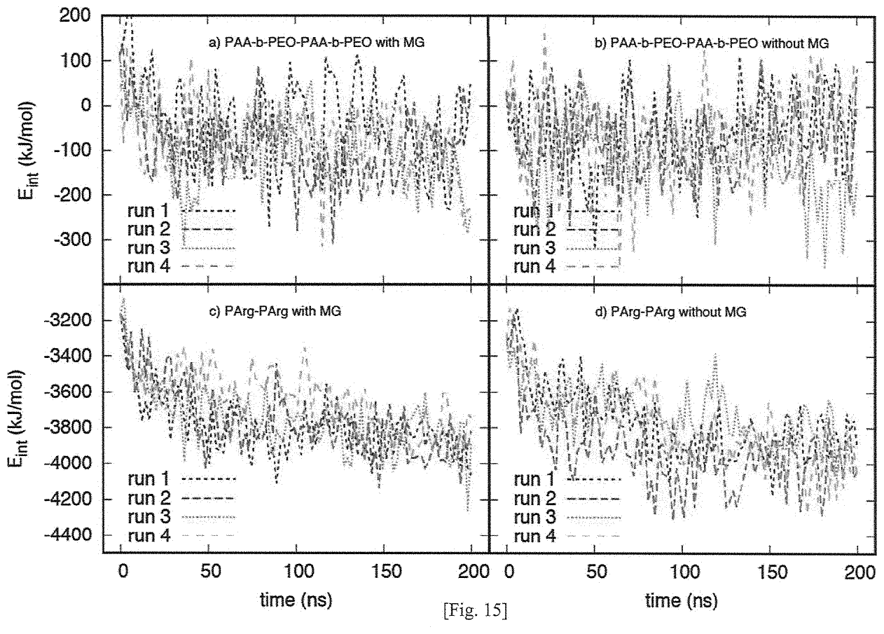

The calculated intramolecular interaction energies are summarized in FIGS. 14 and 15. FIG. 14 indicates that there are strong repulsive interactions within PAA-b-PEO molecules, whereas there are attractive Coulombic interactions between PArg molecules. The first observation can be explained by repulsion between like charges of PAA block and strongly hydrated PEO blocks. The MG molecule seems to have no effect on these interactions. For the PArg molecule (FIG. 14) it is possible to observe the Coulombic interactions are expectedly very favorable throughout the simulations though they seem to become slightly less favorable in the first 100 ns and plateau thereafter. The PArg intramolecular Coulomb interactions also seem slightly more favorable for the MG-containing systems.

Lennard-Jones pair interactions describe interactions between two neutral atoms or molecules, and thus, can be used to determine the hydrophobic properties of the system evaluated. Attractive Lenard-Jones interactions are associated with hydrophobic interactions. The intramolecular Lennard-Jones interactions energies calculated for the systems under investigation are shown in FIG. 15. Overall, the interactions seem to be attractive, though for PAA-b-PEO molecules these interactions are initially repulsive, when PAA-b-PEO chains are in close proximity, which is expected behavior at the beginning of the simulations. Significant energy oscillations were observed in investigated systems that can be explained by time required for polymer chains to adapt the most energetically favorable conformation in solution. For PArg (FIG. 15) the behavior is more predictable, with the interactions becoming more favorable as the simulation progresses, which can be rationalized by the protein folding resulting in exposing hydrophilic groups and burying the hydrophobic backbone atoms. MG seems to be involved in this process and makes it less random (FIG. 13c) and more targeted (FIG. 13d).

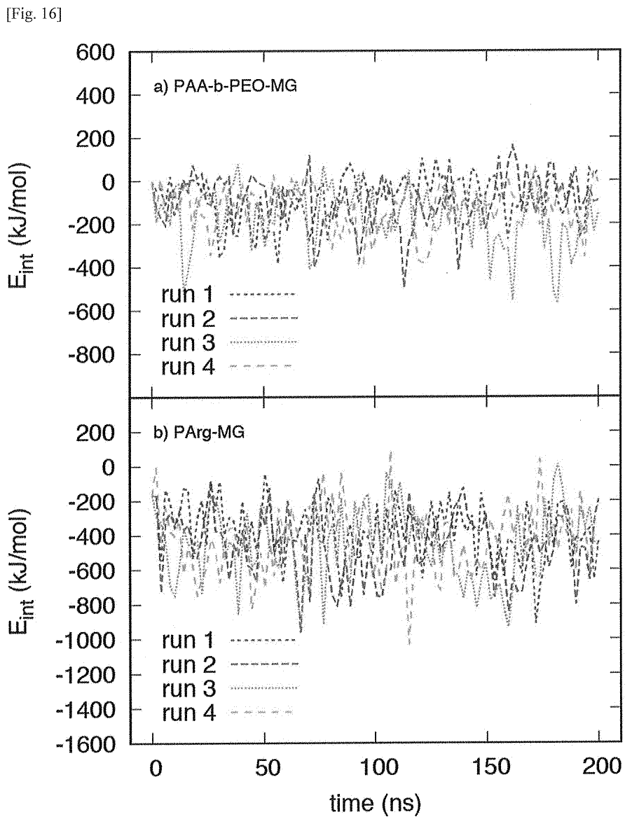

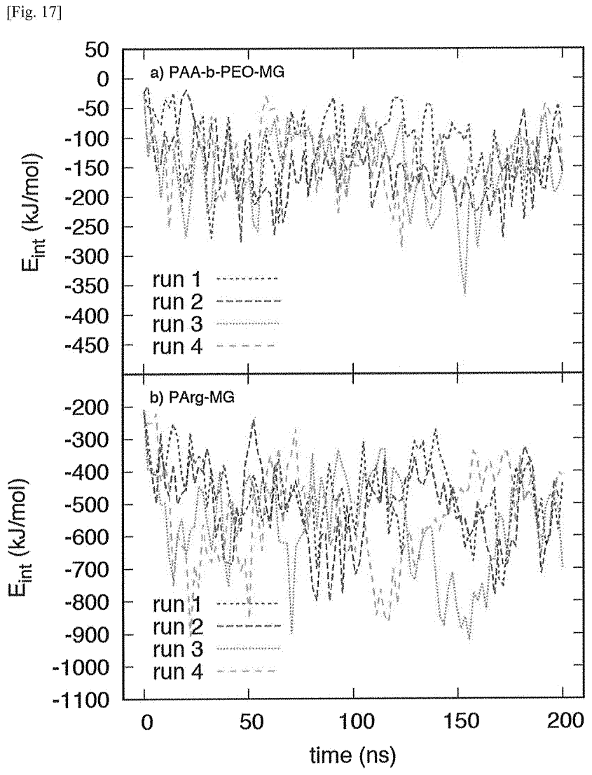

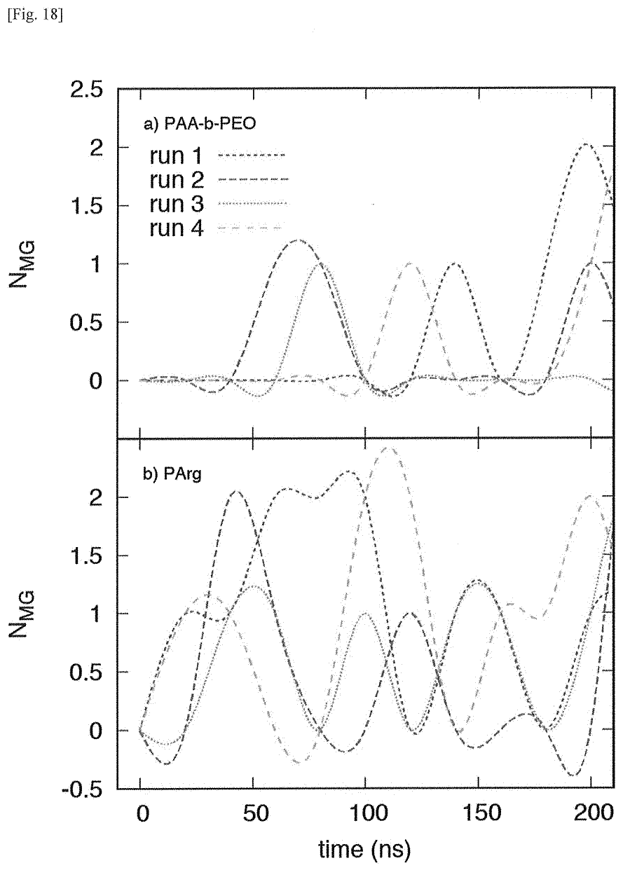

Protein folding in solution is a process best described as a “random walk” because the protein would randomly interact with itself and water molecules, form and break hydrogen bonds with itself and/or water. In a solution containing MG this process is less random. MG can form hydrogen bonds with water thus reducing the number of water molecules available for hydrogen bonding, on top of that, it can also interact with PArg. These two processes significantly lower the number of possible conformations PArg can adopt and is consistent with our observations. This finding appears to be corroborated by the energies of interactions between MG and either PAA-b-PEO or PArg, as shown in FIGS. 16 and 17.

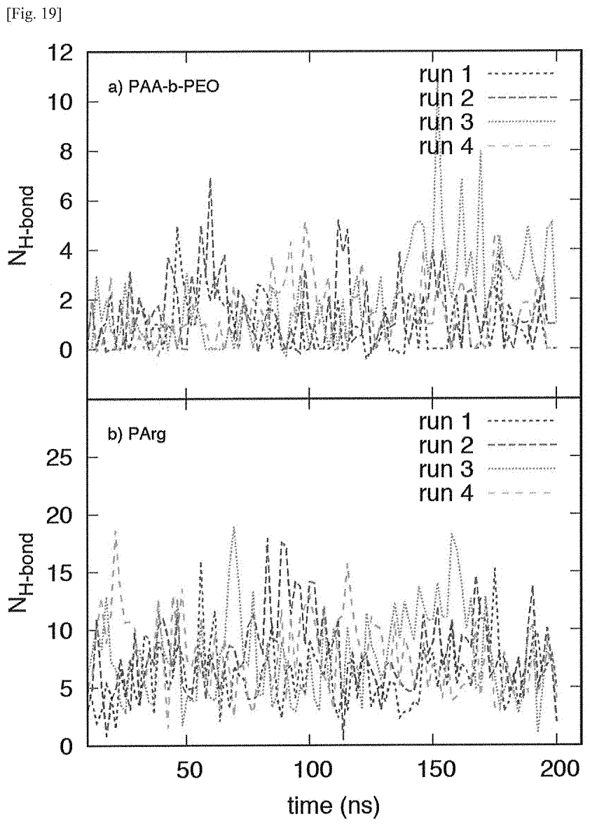

While we find both Coulombic and Lennard-Jones interactions to be attractive for both PAA-b-PEO and PArg, they appear to be substantially more attractive for PArg. This observation was in agreement with statistical analysis of distribution of MG molecules in proximity of PAA-b-PEO and PArg shown in FIG. 16. PArg molecules are more often than not surrounded by MG molecules, while the opposite is true for PAA-b-PEO molecules. The simulations also indicated that there are more hydrogen bonds formed between MG and PArg molecules than between MG and PAA-b-PEO molecules. The results are shown in FIG. 19.

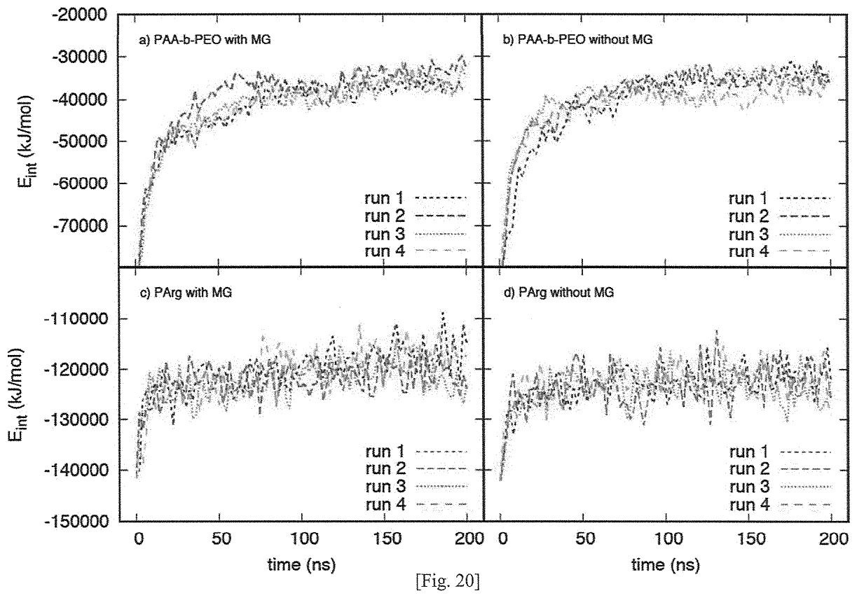

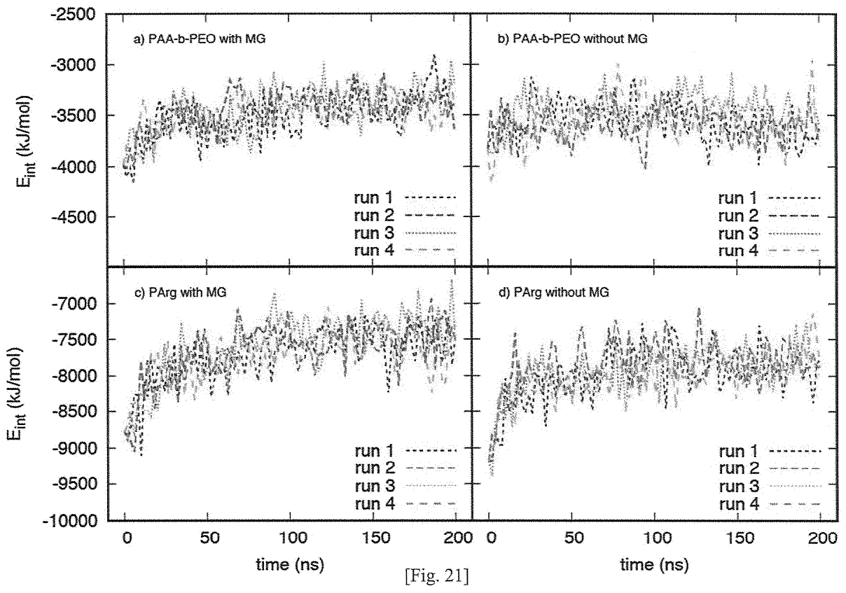

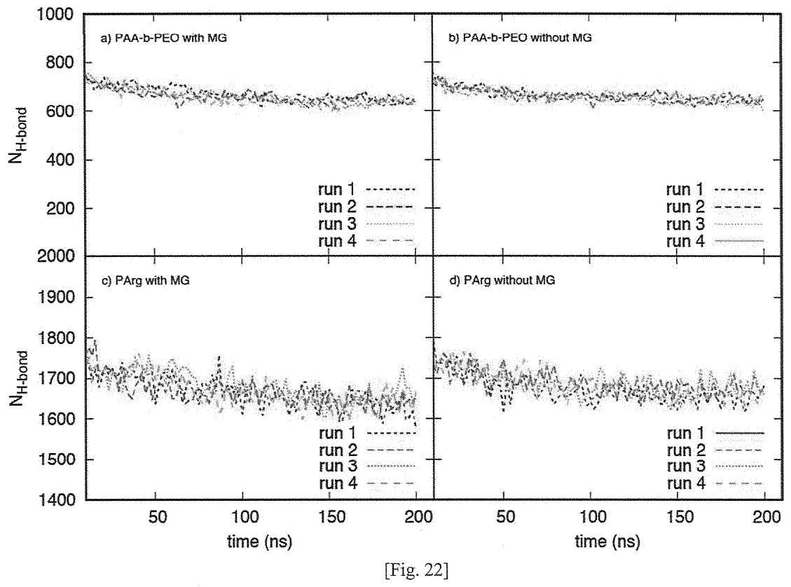

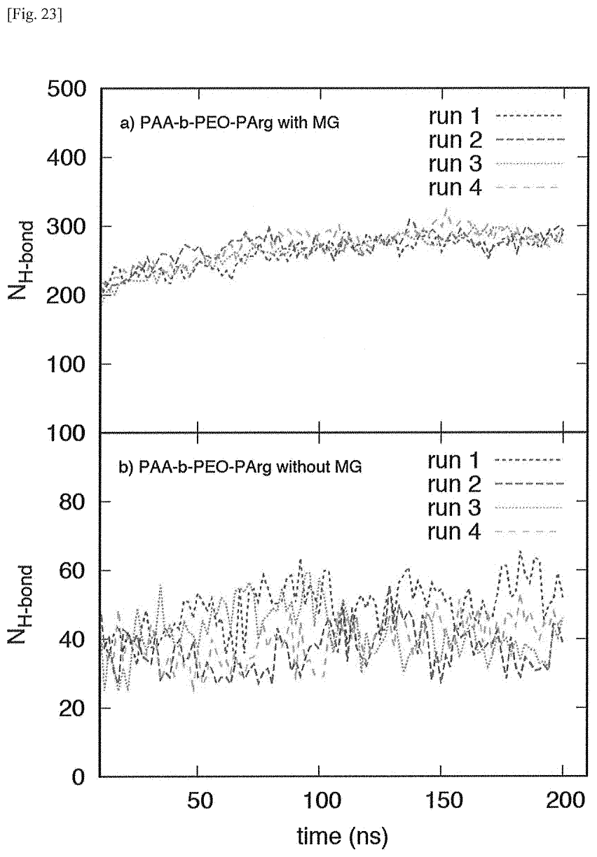

Further insights can be gained by analyzing both the interactions between PAA-b-PEO, PArg and water, and the hydrogen bonding patterns for these pairs. The results discussed so far suggest that the MG molecules interfere with the conformational changes that PArg undergoes in aqueous solution, resulting in partial exposure of hydrophobic moieties to the solvent. If that was indeed the case, less favorable interactions between PArg and water are expected, along with less hydrogen bonds being formed between PArg and water. Data shown in FIGS. 20-22 seem to support this finding.

The simulations were able to detect differences between the conformational changes of PArg in systems with and without MG molecules. In systems without MG the protein folding process appears to follow an almost random-like pattern and not reaching a stable conformation. In systems with present MG molecules, the process becomes less random and leads to some preferred conformation/−s. This is achieved by forming hydrogen bonds with the arginine side chains. It appears that in a mixture of the polyelectrolytes, formation of hydrogen bonds between MG and PArg is more favorable than between MG and PAA-b-PEO molecules. It also appears MG molecules can facilitate the aggregation of PAA-b-PEO and PArg too as evidenced by very few hydrogen bonds between PAA-b-PEO and PArg in systems without MG. Our calculations confirm the stabilizing effect of the MG molecule on ICs system.

### Example 7—Studies on Loading and Release of the Active Compound from ICs

The experimental and theoretical results indicate preference of MG to interact with PArg rather than with PAA-b-PEO block copolymer. Thus, the adapted strategy for the release of MG from ICs was to cleave PArg with Pronase.

The experiment was done according to the following protocol: a fresh solution of ICs in 10 mM NaCl, pH 7 and concentration of approximately 1 mg/ml, at PMC determined during light scattering titrations was prepared. Three groups of 6 samples each were prepared. 6+6 samples (samples labelled “a” and “b”) were duplicates consisting of IC and MG of 6 different concentrations [% w/w]. The remaining 6 samples (control, samples labelled as “c”) contained corresponding amount of MG and volume of solvent (10 mM NaCl) corresponding to the volume of ICs solutions used in other samples.

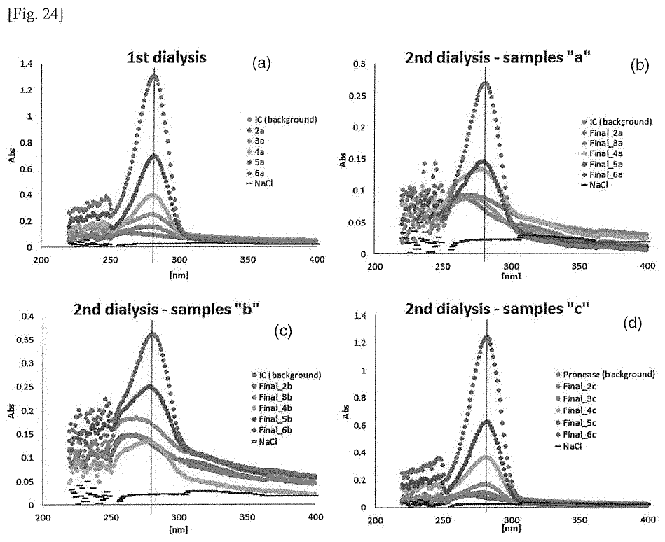

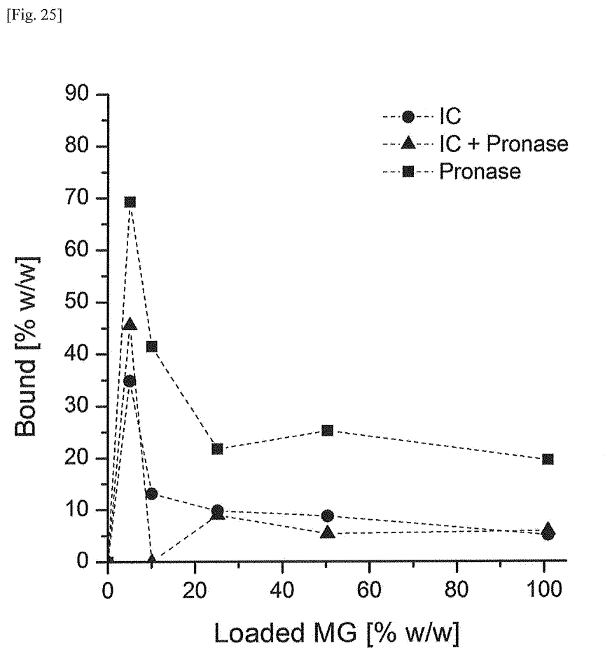

The thus prepared samples were equilibrated for 24 hours at room temperature and subsequently samples containing ICs were dialyzed against the known volume of solvent (10 mM NaCl) under the same conditions. The membrane cut-off was 3.5 k to prevent release of polymers. The concentration of the released MG was determined by means of UV spectroscopy. Subsequently 0.55 ml of solution of Pronase (2 mg/ml) in 10 mM NaCl was added to a set of 6 samples consisting of IC and MG and 6 samples consisting of MG. The remaining 6 samples (ICs+MG) were used as control. The samples were equilibrated at 37° C. under continuous agitation, followed by dialysis against known volume of freshly prepared solvent (10 mM NaCl) for 24 hours at room temperature. The concentration of the released MG was determined by means of UV spectroscopy. The results are shown in FIGS. 24 and 25.

The results indicate no significant difference between the amount of MG released from ICs-MG treated and not treated with Pronase suggesting, that the release was a spontaneous process that takes place due to concentration gradient upon contact with solvent. The results also suggest the increased residue of MG in samples that did not contained ICs and were treated with Pronase. There are two possible explanations to these observations: (i) Pronase binds MG in non-reversible manner, (ii) the measurements contain significant errors due to overlapping signals from ICs, MG and Pronase.

FIG. 24 shows raw UV spectra of samples measured at different stages of the experiment. One may observe that after the first dialysis (prior addition of Pronase) concentration of the released MG was high, and the signal from ICs in the absence of MG is significant at the wavelength corresponding to maximum UV absorption by MG. Similar observation was made during analysis of samples after second dialysis—concentrations of the released MG are less, but wavelength of the maximum UV adsorption by MG corresponds to wavelength at which significant UV absorption by ICs takes place. During data analysis, addition of the signals was assumed and residual signals from ICs and Pronase were subtracted from samples signal. This might have given rise to errors in data analysis.

### INDUSTRIAL APPLICABILITY

The present invention, for the first time, provides a method of loading, storing, transporting, delivering and unloading small, uncharged, non-ionic molecules or compounds using ionomer complexes.

The methods disclosed herein can be performed in a single phase and allows for spontaneous association between the compounds/molecules and the ionomer complex. The methods disclosed herein are reproducible and scalable and lend themselves to useful applications.

For instance, it is envisioned that the disclosed methods (and the ionomer complexes prepared therefrom) may be used for preparing cosmetic or medical compositions, wherein the controlled and targeted delivery of active compounds to a specific unloading site is required. The ionomer complexes disclosed herein may be used in compositions, creams, emulsions, liquids or pills intended for cosmetic or therapeutic skin treatment, in nanomedicine and/or other biomedical applications.

The presently disclosed methods and complexes are especially useful for storing active ingredients whereby the encapsulation, preservation and eventual controlled release of these active ingredients are desired. These may include applications in food science, in coatings and paint chemistry, in agricultural products, pesticidal products, antimicrobial products, etc.

The present application may also find utility in the field of surface coatings in particular, given the knack of ionomer complexes to adhere to solid surfaces. These surface coatings may serve as functionalized surface coatings intended to release one or more active substances after application onto the surface, e.g., insecticides, etc.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A method of coupling an active compound to an ionomer complex, the method comprising at least one step selected from:
(a) mixing a solution comprising at least one polycation and at least one polyanion, with the active compound; or
(b) adding the polycation to a solution comprising the polyanion that is coupled to the active compound; or
(c) adding the polyanion to a solution comprising the polycation that is coupled to the active compound;
to thereby form the ionomer complex having the active compound encapsulated within therein;
wherein the active compound is uncharged and water soluble; and,
wherein the interaction between said active compound and the ionomer complex is non-ionic, and non-covalent in nature.

2. The method of claim 1, wherein the interaction is selected from the group consisting of: hydrogen bonding, hydrophobic interactions, Lenard-Jones interactions, and Van der Waals forces.

3. The method of claim 1, further comprising a step of selecting the polycation and the polyanion for forming the ionomer complex, such that the ionomer complex is capable of partaking in the non-ionic and/or non-covalent interactions with the active compound.

4. The method of claim 1, wherein said active compound has an atomic mass of less than 1 kDa.

5. The method of claim 1, wherein at least one of the polycation or the polyanion is a block co-polymer having a neutral block.

6. The method of claim 5, wherein, in the block copolymer, the ratio of neutral repeating units to charged repeating units is at least 3.

7. The method of claim 1, wherein said ionomer complex comprises one or more functional groups selected from the group consisting of: an alcohol group, a carbonyl group, an ether group, an ester group, a carboxylic acid group, an amine group, an amide group, a carbamide group, an imine group, an imino group, an imidazole group, a guanidine group, a fluoro group and a cyano group, and wherein said one or more functional groups is coupled to the active compound by the non-ionic and non-covalent interaction.

8. The method of claim 1, wherein the polyanion is selected from the group consisting of: polyacrylic acid, polymethacrylic acid, polystyrene sulfonate, polyphosphoric acid, polyglutamic acid and polyaspartic acid.

9. The method of claim 1, wherein the polycation is selected from the group consisting of: poly-L-arginine, poly-D-arginine, poly-L-tryptophan, poly-D-tryptophan, poly-L-histidine, poly-D-histidine, α-poly-L-lysine, ε-poly-L-lysine, α-poly-D-lysine and ε-poly-D-lysine.

10. The method of claim 5, wherein said neutral block is selected from the group comprising polyethylene oxide (PEO), polytyrosine, polylactic acid, polycaprolactone, polyurethanes or polyanhydrides.

11. An ionomer complex comprising at least one active compound coupled thereto,
wherein said active compound is uncharged and water-soluble, and
wherein the active compound is coupled to the ionomer complex via a non-ionic, and non-covalent interaction.

12. The ionomer complex of claim 11, wherein said active compound has an atomic mass of less than 1 kDa.

13. The ionomer complex of claim 11, wherein said ionomer complex is formed from a reaction between a polycation and a polyanion, wherein at least one of the polycation or the polyanion is a block co-polymer having a neutral polymer block.

14. The ionomer complex of claim 13, wherein, in the block co-polymer, the ratio of neutral repeating units to charged repeating units is at least 3.

15. The ionomer complex of claim 13, wherein at least one of said polycation or polyanion or the ionomer complex comprises functional groups capable of coupling to the active compound via non-ionic and non-covalent interactions.

16. The ionomer complex of claim 13, wherein each repeating group of the polyanion or of the polyanion comprises one or more functional groups selected from the group consisting of: an alcohol group, a carbonyl group, an ether group, an ester group, a carboxylic acid group, an amine group, an amide group, a carbamide group, an imine group, an imino group, an imidazole group, a guanidine group, a fluoro group and a cyano group, said functional group being capable of coupling to said active compound via the non-ionic, and non-covalent interaction.

17. The ionomer complex of claim 13, wherein the polyanion is selected from the group comprising polyacrylic acid, polymethacrylic acid, polystyrene sulfonate, polyphosphoric acid, polyglutamic acid or polyaspartic acid.

18. The ionomer complex of claim 13, wherein the polycation is selected from the group comprising poly-L-arginine, poly-D-arginine, poly-L-tryptophan, poly-D-tryptophan, poly-L-histidine, poly-D-histidine, α-poly-L-lysine, ε-poly-L-lysine, α-poly-D-lysine or ε-poly-D-lysine.

19. The ionomer complex of claim 13, wherein said neutral block is selected from the group comprising polyethylene oxide (PEO), polytyrosine, polylactic acid, polycaprolactone, polyurethanes or polyanhydrides.
